(19) United States
(12) Patent Application Publication
(10) Pub. No.: US 2024/0257904 A1
(43) Pub. Date: Aug. 1, 2024

(54) COPY NUMBER VARIANT CALLING AND RECOVERY

(71) Applicant: Illumina, Inc., San Diego, CA (US)

(72) Inventors: Eric Roller, San Diego, CA (US); Aaron Halpern, San Diego, CA (US); Sean Truong, San Diego, CA (US)

(73) Assignee: Illumina, Inc., San Diego, CA (US)

(21) Appl. No.: 18/421,362

(22) Filed: Jan. 24, 2024

Related U.S. Application Data
(60) Provisional application No. 63/482,451, filed on Jan. 31, 2023.

(51) Int. Cl. G16B 20/10; G16B 30/00; G16B 50/30

(52) U.S. Cl. CPC G16B 20/10; G16B 30/00; G16B 50/30

(57) ABSTRACT

Improved copy number variant (CNV) calling in a genomic sequence, and potential recovery, includes (i) obtaining genetic sequence variant data that includes records indicating structural variant(s) (SVs) and records indicating CNV(s) in the genomic sequence, (ii) determining, based on an initial CNV indicated in the genetic sequence variant data and on initial SV(s) indicated in the genetic sequence variant data, an SV-informed CNV call as an updated version of the initial CNV, where the determining uses information from the initial SV(s) to determine a start breakpoint position and an end breakpoint position for the SV-informed CNV call, at least one of the start breakpoint position and end breakpoint position being updated, informed by the initial SV(s), in comparison to a corresponding start breakpoint position and/or end breakpoint position of the initial CNV, and (ii) writing the determined SV-informed CNV call as record(s) in a genetic sequence variant data file.

### BACKGROUND

In the field of nucleic acid sequencing, a structural variant (SV) is a relatively large genomic variation found in an individual's genomic deoxyribonucleic acid (DNA). Variations are determined relative to a reference sequence. Copy number variants/variations (CNVs)—a subset of SVs—are structural variations in base pairs (bp) of genetic material where large sections of the genome are duplicated or deleted. Variation in DNA copy number is a well-described cause of human genetic disease. CNV detection technology progressed from karyotyping or microarray-based clinical diagnostic tests, with lengths on the order of kilobases to megabases, to next-generation sequencing (NGS), which has provided advancements in sequencing technology, including technology for more accurate CNV calling.

### SUMMARY

Shortcomings of the prior art are overcome and additional advantages are provided through the provision of a computer-implemented method for improved calling of copy number variants in a genomic sequence. The method includes obtaining genetic sequence variant data that includes records indicating at least one structural variant (SV) and records indicating at least one copy number variant (CNV) in the genomic sequence. The method also determines, based on an initial CNV indicated in the genetic sequence variant data and on at least one initial SV indicated in the genetic sequence variant data, an SV-informed CNV call as an updated version of the initial CNV, where the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV. The method further writes the determined SV-informed CNV call as one or more records in a genetic sequence variant data file.

Further, a computer system is provided that includes a memory and a processor in communication with the memory, wherein the computer system is configured to perform a computer-implemented method for improved calling of copy number variants in a genomic sequence. The method includes obtaining genetic sequence variant data that includes records indicating at least one structural variant (SV) and records indicating at least one copy number variant (CNV) in the genomic sequence. The method also determines, based on an initial CNV indicated in the genetic sequence variant data and on at least one initial SV indicated in the genetic sequence variant data, an SV-informed CNV call as an updated version of the initial CNV, where the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV. The method further writes the determined SV-informed CNV call as one or more records in a genetic sequence variant data file.

Yet further, a computer program product including a computer readable storage medium readable by a processing circuit and storing instructions for execution by the processing circuit is provided for performing a computer-implemented method for improved calling of copy number variants in a genomic sequence. The method includes obtaining genetic sequence variant data that includes records indicating at least one structural variant (SV) and records indicating at least one copy number variant (CNV) in the genomic sequence. The method also determines, based on an initial CNV indicated in the genetic sequence variant data and on at least one initial SV indicated in the genetic sequence variant data, an SV-informed CNV call as an updated version of the initial CNV, where the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV. The method further writes the determined SV-informed CNV call as one or more records in a genetic sequence variant data file.

In one or more embodiments, the initial CNV is provided by a CNV calling component of genomic analysis software. In one or more embodiments, the genomic analysis software has a filtering component configured to filter-out CNVs of which a confidence level is less than a threshold confidence level, the CNV calling component provides a confidence level of the initial CNV that is less than the threshold confidence level, and the SV-informed CNV call is provided with a confidence level higher than the threshold confidence level such that the filtering component does not filter-out the SV-informed CNV call.

In one or more embodiments, a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit. In one or more embodiments, a breakpoint resolution of the SV-informed CNV call is 1 base pair.

In one or more embodiments, the determining the SV-informed CNV call as the updated version of the initial CNV includes modifying a record of the initial CNV to produce a record of the SV-informed CNV call. The modifying can change the start and/or end breakpoint positions of the initial CNV, as indicated in the record of the initial CNV, to be the determined updated breakpoint position(s) informed by the at least one initial SV, and can further update a length of the CNV indicated in the record of the initial CNV and a quality score in the record of the initial CNV, to provide the record of the SV-informed CNV call.

In one or more embodiments, the record of the initial CNV is a copy of an original record of the initial CNV, where the genetic sequence variant data file is part of one or more genetic sequence variant data files, and where the original record of the initial CNV is retained and output in at least one of the one or more genetic sequence variant data files.

In one or more embodiments, determining the SV-informed CNV call includes performing, for each initial SV of the at least one initial SV, a pairwise comparison of the initial CNV to the initial SV.

In one or more embodiments, the pairwise comparison of the initial CNV to the initial SV includes one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.

In one or more embodiments, the one or more breakpoint comparisons include at least one of: comparing a start breakpoint position of the initial CNV to at least one of the start breakpoint position or end breakpoint position of the initial SV; or comparing an end breakpoint position of the initial CNV to at least one of the start breakpoint position or end breakpoint position of the initial SV.

In one or more embodiments, the one or more rules include at least one of: a rule requiring at least some positional overlap between the initial SV and the proposed modified CNV; a rule for compatibility in orientation of the initial SV and the proposed modified CNV; a rule for correlated breakpoints of the initial SV and proposed modified CNV to be within a threshold distance; or a rule for uniqueness requiring that a breakpoint, of the initial CNV, proposed for modification match to at most one SV breakpoint of the at least one initial SV.

In one or more embodiments, a length of the SV-informed CNV call is less than or equal to 20,000 base pairs. In one or more embodiments, a length of the SV-informed CNV call is less than or equal to 10,000 base pairs. In one or more embodiments, a length of the SV-informed CNV call is less than a length of the initial CNV. In one or more embodiments, the length of the initial CNV is less than or equal to 20,000 base pairs.

Additional features and advantages are realized through the concepts described herein.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts example structural variant types;

FIG. 2 depicts example copy number variant duplication and deletion events;

FIG. 3 depicts an example copy number variant calling workflow;

FIG. 4 depicts an example structural variant calling workflow;

FIGS. 5-6 depict example accuracies in copy number variant and structural variant deletion calling against a benchmark dataset;

FIG. 7 depicts example accuracies in copy number variant deletion calling based on structural-variant-informed copy number variant calling in accordance with aspects described herein;

FIG. 8 depicts a comparison graph of structural variant, copy number variant, and structural-variant-informed copy number variant calling in accordance with aspects described herein;

FIG. 9 depicts a conceptual representation of misaligned breakpoints of a structural variant call and copy number variant call corresponding to the same event;

FIG. 10 presents an example interface illustrating a comparison between a structural-variant-informed copy number variant call in accordance with aspects described herein and the associated copy number variant-only and structural variant-only calls from conventional copy number variant and structural variant call processing;

FIG. 11 presents another example interface illustrating a comparison between a structural-variant-informed copy number variant call in accordance with aspects described herein and the associated copy number variant-only and structural variant-only calls from conventional copy number variant and structural variant call processing;

FIG. 12 presents a further example interface illustrating a comparison between a structural-variant-informed copy number variant call in accordance with aspects described herein and the associated copy number variant-only and structural variant-only calls from conventional copy number variant and structural variant call processing;

FIG. 13 depicts an example conceptual diagram of CNV and SV calls along a segment of a genome;

FIG. 14 depicts a conceptual diagram of a process for structural-variant-informed copy number variant calling and output, in accordance with aspects described herein;

FIG. 15 depicts a depth and breakend profile of an example scenario for SV-informed CNV calling in accordance with aspects described herein;

FIG. 16 presents example possible reconstructions of the event depicted in FIG. 15 in accordance with aspects described herein;

FIG. 17 depicts another depth and breakend profile of an example scenario for SV-informed CNV calling in accordance with aspects described herein;

FIGS. 18A-18C depict additional example scenarios for SV-informed CNV calling in accordance with aspects described herein;

FIGS. 19A-19B depict example processes for improved calling of copy number variants in a genomic sequence, in accordance with aspects described herein; and

FIG. 20 depicts an example of a computer system and associated devices to incorporate and/or use aspects described herein.

### DETAILED DESCRIPTION

There have been challenges and shortcomings in the detection and accurate calling of ‘short’ CNVs. While ‘short’ in this context is not a fixed number, it typically up to about 20 kilobase pairs (kbp). For this reason, many conventional approaches filter out short CNV events smaller than some configurable number, usually in the 10 kbp to 20 kbp range, due to low accuracy. While NGS has provided some improvement in short CNV detection, there is a need for better CNV calling, and particularly for short CNVs. For purposes of explanation and description herein, a ‘short’ CNV is one that is <10 kbp in length.

Thus, described herein are approaches for improved CNV calling that uses SV call data to inform CNV call modifications. This can result in potential recovery of CNVs that otherwise would have been filtered-out from reporting. Improved CNV calling as described herein provides valuable copy number information that is not otherwise available from an SV caller. SV callers, unlike CNV callers, generally do not include a copy number assignment, but such information can be critical to genomic analysis and therefore accurate identification and calling of CNVs as such, including short CNVs, is useful. Further provided is increased precision and confidence in the specific locations of copy number changes. SV calling alone has historically lacked the accuracy needed for some clinical applications. Conventional CNV calling partially addresses this limitation but, as noted, has been limited by low confidence levels on short CNV calls. Aspects described herein provide for integration of SV call data and CNV call data to enable a CNV caller to provide more accurate CNV calls with higher confidences.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4- cthanocytosine, 2,6-diaminopurine, N6-ethano-2,6-diaminopurine, 5-methylcytosine, 5-(C3-C6)- alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, 5-propynyl dCTP, and 5-propynyl-dUTP.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference sequence is that of a full-length genome. Such sequences may be referred to as genomic reference sequences. For example, the reference sequence can be a reference human genome sequence, such as hg19 or hg38. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” or “medical condition” is used herein as a broad term that includes all diseases and disorders, but can include injuries and normal health situations, such as pregnancy, that might affect a person's health, benefit from medical assistance, or have implications for medical treatments.

As used herein, the term “cluster” or “clump” refers to a group of molecules, e.g., a group of DNA, or a group of signals. In some embodiments, the signals of a cluster are derived from different features. In some embodiments, a signal clump represents a physical region covered by one amplified oligonucleotide. Each signal clump could be ideally observed as several signals. Accordingly, duplicate signals could be detected from the same clump of signals. In some embodiments, a cluster or clump of signals can comprise one or more signals or spots that correspond to a particular feature. When used in connection with microarray devices or other molecular analytical devices, a cluster can comprise one or more signals that together occupy the physical region occupied by an amplified oligonucleotide (or other polynucleotide or polypeptide with a same or similar sequence). For example, where a feature is an amplified oligonucleotide, a cluster can be the physical region covered by one amplified oligonucleotide. In other embodiments, a cluster or clump of signals need not strictly correspond to a feature. For example, spurious noise signals may be included in a signal cluster but not necessarily be within the feature area. For example, a cluster of signals from four cycles of a sequencing reaction could comprise at least four signals.

The term “next generation sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene. For example, a sequence read may be a short string of nucleotides (e.g., 20-150 bases) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50x, 100x, etc., where “x” refers to the number of times a locus is covered with a sequence read. Sequencing depth can also be applied to multiple loci, or the whole genome, in which case x can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100×in sequencing depth.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc. In some cases, “effective read coverage” of a chromosome is defined as the actual amount of bases covered by reads. Sequencing depth, which refers to the expected coverage of nucleotides by reads, is computed based on the assumption that reads are synthesized uniformly across chromosomes. In reality, read coverage across genomes is not uniform. Although a coverage of 10x, for example, means a nucleotide is covered 10 times on average, in certain parts of a genome, nucleotides are covered much more or much less. One factor that influences coverage is the ability of a read aligner to align reads to genomes. If a part of a genome is complex, e.g. having many repeats, aligners might have troubles aligning reads to that region, resulting in low coverage.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, DRAGEN, drFAST, ELAND, ERNE, GNUMAP, GEM, GensearchNGS, GMAP and GSNAP, Gencious Assembler, LAST, MAQ, mrFAST and mrsFAST, MOM, MOSAIK, MPscan, Novoalign & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The term “mapping” used herein refers to specifically assigning a sequence read to a larger sequence, e.g., a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (e.g., ancuploidy), partial chromosome abnormality or mosaicism, each of which is described in greater detail herein. Non-limiting examples of genetic variations include one or more deletions (e.g., micro-deletions), duplications (e.g., micro-duplications), insertions, mutations, polymorphisms (e.g., single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair to about 250 megabases (Mb) in length. In some embodiments, an insertion, repeat, deletion, duplication, mutation or polymorphism is about 1 base or base pair to about 1,000 kilobases (kb) in length (e.g., about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (e.g., a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (e.g., a genetic aberration) in which a part of a chromosome or a sequence of DNA is copied and inserted back into the genome. In certain embodiments a genetic duplication (i.e. duplication) is any duplication of a region of DNA. In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e. insertion) of a single base.

A genetic variation sometimes includes copy number variations (CNVs), i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

As used herein, the term “short CNV” refers to a CNV that is less than ten-thousand base pairs in length, i.e. <10 kbp.

As used herein, the term “array” may refer to a sequence of given size in the genome.

As used herein, the term “copy number” refers to the number of times (e.g., 0, 1, 1.5, 2, 3.5, 5, etc.) the repeat unit is repeated. The change in copy number can be represented as the difference in copy number relative to the reference (e.g., −1, 0, +1, +2, etc.).

As used herein, the term “fragment size” refers to the length of the original nucleic acid sequence used to generate paired-end reads, calculated based on where those reads are mapped.

As used herein, the term “indels” refers to small insertions or deletions less than 50 base pairs in length in a nucleic acid sequence.

As used herein, the term “paired-end reads” or “paired end reads” refers to paired reads generated from sequencing the forward and reverse ends of a larger nucleic acid fragment. In some examples, the forward and reverse ends of a larger nucleic acid fragment may share the same name. The paired-end reads may be generated from paired end sequencing that obtains one read from each end of a nucleic acid fragment.

As used herein, the term “pattern” refers to the sequence of a repeat unit of the tandem repeat.

As used herein, the term “mate” or “mate of a read” refers to the pair of the read in question; i.e., the other read generated from the same nucleic acid fragment.

As used herein, the term “repeat unit” refers to the sequence of a single copy that is repeated multiple times.

As used herein, the term “single nucleotide variants” or “SNVs” refers to single base substitutions in a nucleic acid sequence.

As used herein, the term “small variant event” refers to a collection of adjacent SNVs or indels that occurs in the same haplotype array within a maximal distance of each other (for example, a maximal distance of 10 base-pairs).

As used herein, the term “structural variation” or “SV” refers to a nucleic acid variant greater than 50 base pairs corresponding to, e.g. a duplication, deletion, insertion, inversion, or translocation, as examples.

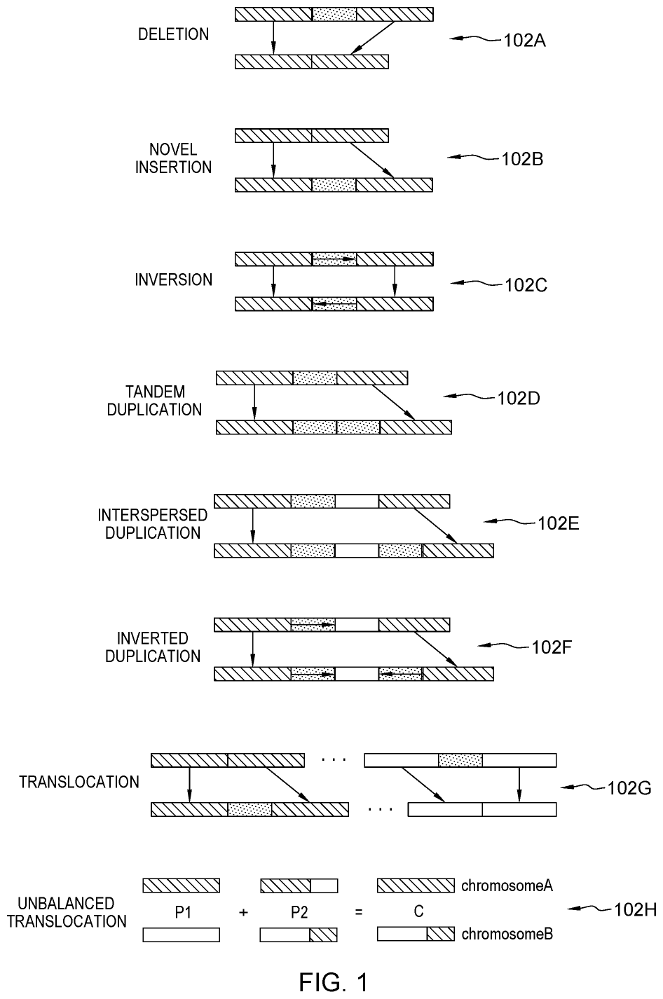

FIG. 1 depicts example structural variant types. The events labeled 102A through 102H depict the following SV types, respectively: deletion, novel insertion, inversion, tandem duplication, interspersed duplication, inverted duplication, translocation, and unbalanced translocation. The stippled sequence in each example 102A-102G is the sequence being deleted, inserted, duplicated, translocated, or inverted. In examples 102A through 102G, a comparison is shown between a reference sequence (on top) and a sample sequence (below the reference sequence). In example 102H, a compound event is shown that leads to an unbalanced translocation.

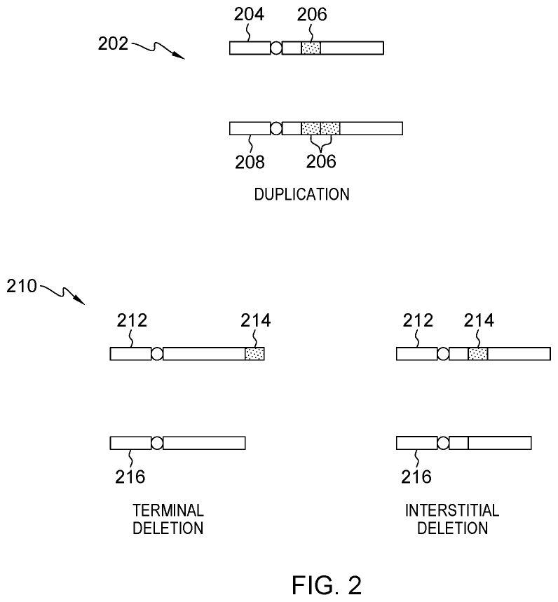

A CNV is either a duplication or a deletion event. FIG. 2 depicts example CNV duplication and deletion events. In a duplication event (202), a sequence 206 of the reference genome 204 is duplicated in the sample sequence 208. In a deletion scenario (210), a sequence 214 of the reference genome 212 is deleted (does not appear) in the sample 216. A terminal deletion is a deletion of the sequence 214 at a terminal portion of the reference genome, while an interstitial deletion is a deletion of the sequence 214 from an interior portion of the reference genome.

CNV calling typically uses a depth-based approach to segment a genome into regions of contiguous germline copy numbers. Under this approach, the genome is stratified into windows (“bins”), and a read depth is measured in ‘bins’. The window size is tailorable. In examples, the windows size is about 1,000 bp long.

Window, or bin, size corresponds to the concept of breakpoint resolution in CNV calling. A breakpoint (also referred to herein as a “breakend” or BND) is a specific base location, within a genome, where an event (e.g. variant) occurs. Breakpoints may be used to indicate a specific base pair location designating the start of the event, and sometimes a location of the end of the event. BND is a mandate of the known variant call format (VCF) specification for specifying the format of text files used for storing sequence variations. While BND notation is robust for use in specifying more complex variants like inversions and translocations, deletion events (DELs) and duplication events (DUPs) can be annotated by BNDs, for instance BNDs for the start and end locations of the event.

Historically, breakpoint location identification was not particularly important; it was sufficient to know merely that a CNV event is present on a particular gene (which could span hundreds of thousands of bases), without needing to know the specific start and end locations of the event. This remains true in some current applications. In other applications, however, the exact location of CNV events are more important to know. The resolution of breakpoints under a binning approach is a function of the window size. With 1 kbp intervals, specific locations are identified by the window where the location is determined to exist, even if not known exactly. The start (or end, or some other singular bp position) of the window might be used as an event start or endpoint (breakend), and the exact location of the start or end of the event could therefore be off by up to approximately the windows size, e.g., 1 kbp. As noted, because of issues surrounding confidence and accuracy of CNV calling, some CNV callers filter out events of length less than some defined threshold, for instance 10 kbp.

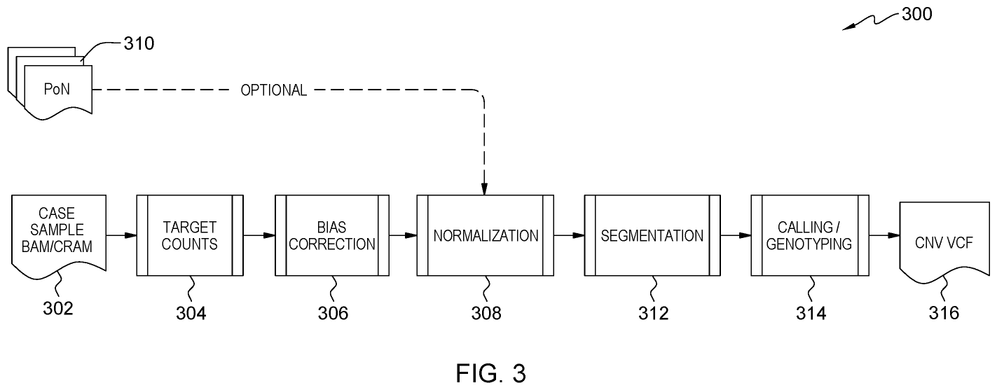

FIG. 3 depicts an example CNV calling workflow. The workflow is a pipeline that may be performed by a genomic sequencing data analysis platform, for instance the DRAGEN® platform available from Illumina Inc, San Diego, California, USA (of which DRAGEN is a registered trademark). Referring to FIG. 3, the workflow 300 begins with input of a case sample (for instance in the BAM/CRAM format) 302 to a target counts processing module 304 that performs binning of the read counts and other signals from the alignments indicated by the case sample. The workflow 300 proceeds to a bias correction processing module 306 that provides correction of intrinsic system biases, then to a normalization processing module 308 for detection of normal ploidy levels and normalization of the case sample. The normalization module 308 can optionally take in a panel of normals (PoN 310), which might be used when a cohort or population samples are readily available. The workflow 300 continues with a segmentation processing module 312 that performs breakpoint detection via segmentation of the normalized signal, followed by calling/genotyping processing module 314 for thresholding, scoring, qualifying, and filtering of putative events as copy number variants. The workflow then outputs results (e.g. records for CNV calls) as file(s) 316 in a given format, for instance the known VCF (i.e., as*. vcf files).

CNV calls by some CNV callers are made by deconstructing events into constituent BND states and then distinguishing between the CNVs and SVs. In this regard, structural variant calls by an SV caller are typically also identified using the BND format. Unlike a read-depth and binning approach taken with CNV calling where read depth is used to detect a change in coverage level, SV calling can leverage split reads and improper reads, with re-assembly of candidate contigs (set of DNA segments or sequences that overlap in a way that provides a contiguous representation of a genomic region). An example SV caller attempts to detect when there is clipping in the alignment. A ‘soft clipping’ in the alignment indicates that some fraction or portion of a read does not belong in the given locus but instead belongs in another part of the chromosome or perhaps another chromosome entirely. The split read aspect provides the ability to determine that a given read is split across two different regions in the genome to give an indication that there is some kind of structural variation that is occurring. The improper read aspect (since using paired end reads with a known fragment length) identifies anomalous events when aligning to the reference sequence, with re-assembly of candidate contigs integrating the reads within the given locus to generate final indication of SV events.

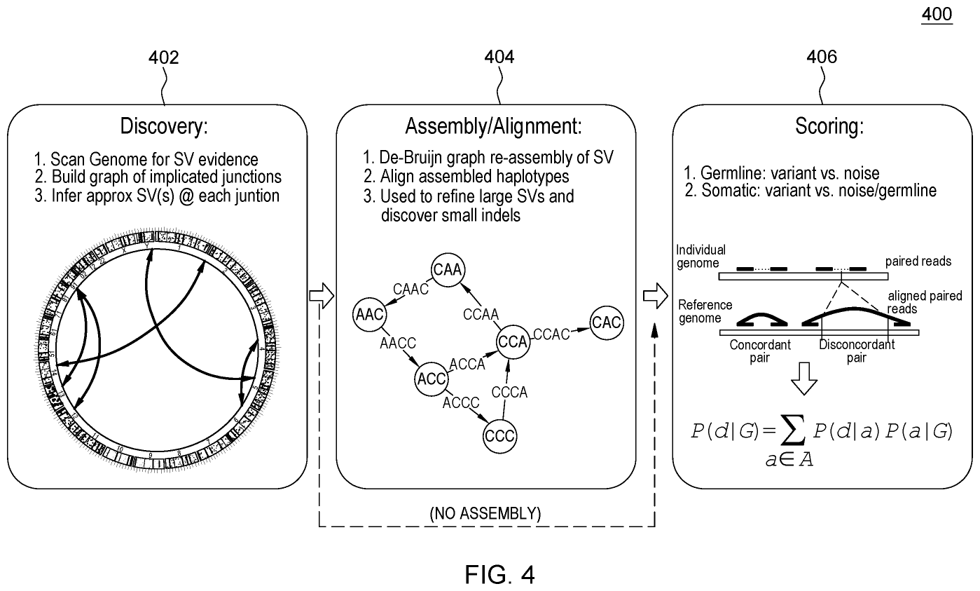

SV calling, unlike CNV calling, has base pair-level breakpoint resolution because of the soft clipping identification and checking at the base pair level. FIG. 4 depicts an example structural variant calling workflow. The workflow 400 proceeds with a discovery stage 402 that scans the genome for SV evidence, builds a graph of implicated junctions, and infers approximate SV(s) at each junction, an assembly/alignment stage 404 that performs De-Bruijn graph reassembly of the SVs, aligns assembled haplotypes, and refines large SVs and discovers small indels, and a scoring stage 406 that performs scoring for germline and somatic variants.

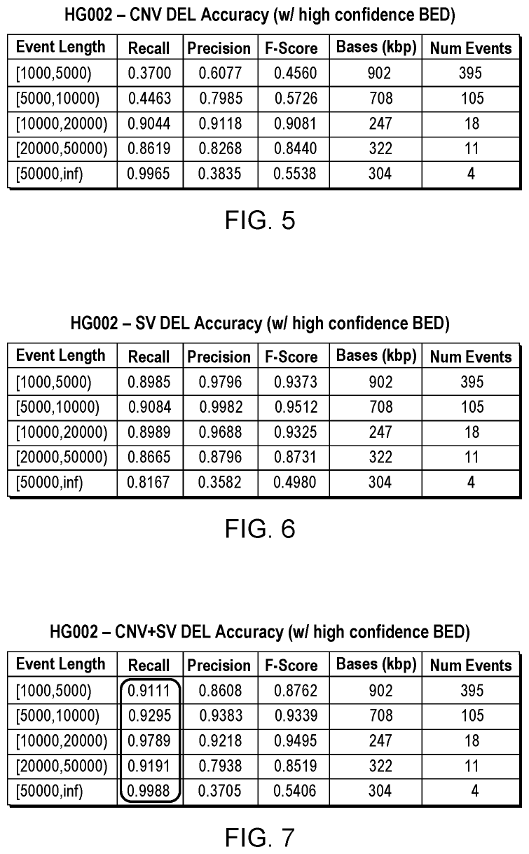

To illustrate an example of differences in CNV and SV call accuracy, FIGS. 5-6 depict example accuracies in CNV and SV deletion calling against a benchmark dataset. Each of FIG. 5 and FIG. 6 presents a table with columns for event length, recall, precision, f-score, bases (in kilo base pairs), and number of events. Each table has rows for different event length ranges in base pairs, i.e., (i) 1000 up to 5000, (ii) 5000 up to 10,000, (iii) 10,000 up to 20,000, (iv) 20,000 up to 50,000, and (v) 50,000 and above. The benchmarking was against regions of high-confidence in the GRCh37 human genome assembly benchmark dataset published by the National Institute of Science and Technology (NIST). With reference to FIG. 5, of particular note are the recall and precision values for relatively short (i.e. less than 10 kpb) CNV calls, particularly as compared to the recall and precision values for SV calls of the same length as shown by FIG. 6. The recall and precision values for CNV events of length 1000 to 5000 were 0.3700 and 0.6077, respectively, and for events of length 5000 to 10000 were 0.4463 and 0.7985, respectively. In contrast, recall and precision values for SV events of lengths 1000 to 5000 were 0.8985 and 0.9796, respectively, and for events of length 5000 to 10000 were 0.9084 and 0.9982, respectively.

As described above, conventional CNV calling, due to the relatively high CNV breakpoint resolution (e.g., ˜1 kbp) and/or other reasons, might filter events smaller than some length (such as 10 kbp) due to such low accuracy as reflected by FIG. 5. However, aspects described herein present a joint CNV-SV calling—SV-informed CNV calling—to improve CNV detection and calling, which provides significant improvements in short CNV (<10 kbp) recall and precision. FIG. 7 depicts example accuracies in CNV deletion calling based on SV-informed CNV calling in accordance with aspects described herein. While improvement is shown for all CNV length ranges, of particular note are CNV events of length<10 kbp, where recall and precision increased dramatically compared to the accuracies shown in FIG. 5.

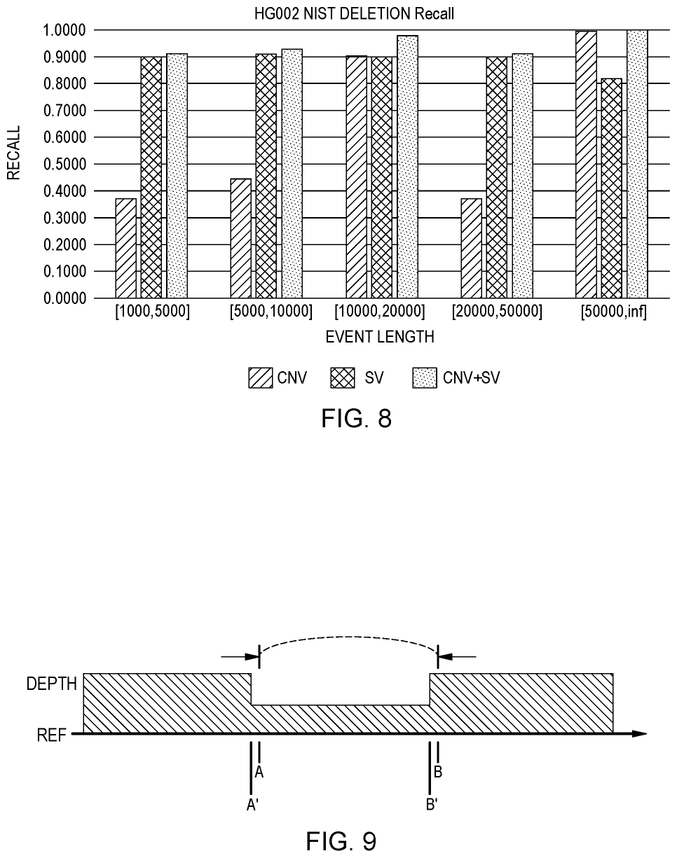

FIG. 8 depicts a comparison graph of structural variant, copy number variant, and structural-variant-informed copy number variant calling, in accordance with aspects described herein. Specifically, the graph compares the recall values from the tables of FIGS. 5, 6, and 7, for each of the 5 event length ranges given, and reflects a dramatic improvement in recall relative to conventional CV calling.

In accordance with aspects of SV-informed CNV calling described herein, breakpoints of one or more identified CNVs (including those that may have initially been labeled for filtering out) are adjusted to align with breakpoints of matching SV calls. Rather than CNV breakpoints being identified based on coverage bin boundaries, breakpoint information from SV call(s), which information has base pair level resolution, is used to more accurately identify the positions and length of called CNVs. This results in CNVs with base pair-level accuracy from the support of the SV caller information that has base pair-level resolution. The quality/confidence of SV-informed CNV events is higher because of the breakpoint signals leveraged from the SV caller, and therefore some CNV calls that would otherwise have been filtered out due to low confidence, i.e. if coverage bin boundary was used for breakpoint identification, could be ‘rescued’ and provided in the output CNV VCF.

FIG. 9 depicts a conceptual representation of misaligned breakpoints of an SV call and CNV call corresponding to the same event (a DEL). The SV breakpoints are indicated in the SV call data with associated confidence intervals that present positional ranges (left-to-right; lowest-to-highest) for the breakpoints to account for uncertainty from the SV caller about exactly where those boundaries are. In FIG. 9, the SV call indicates breakpoints at positions A and B. These can be expected to differ (not exactly align) with the positions (indicated by A′, B′) indicated in the CNV call, which are determined based on changes in read depth under the windowing/binning approach discussed above.

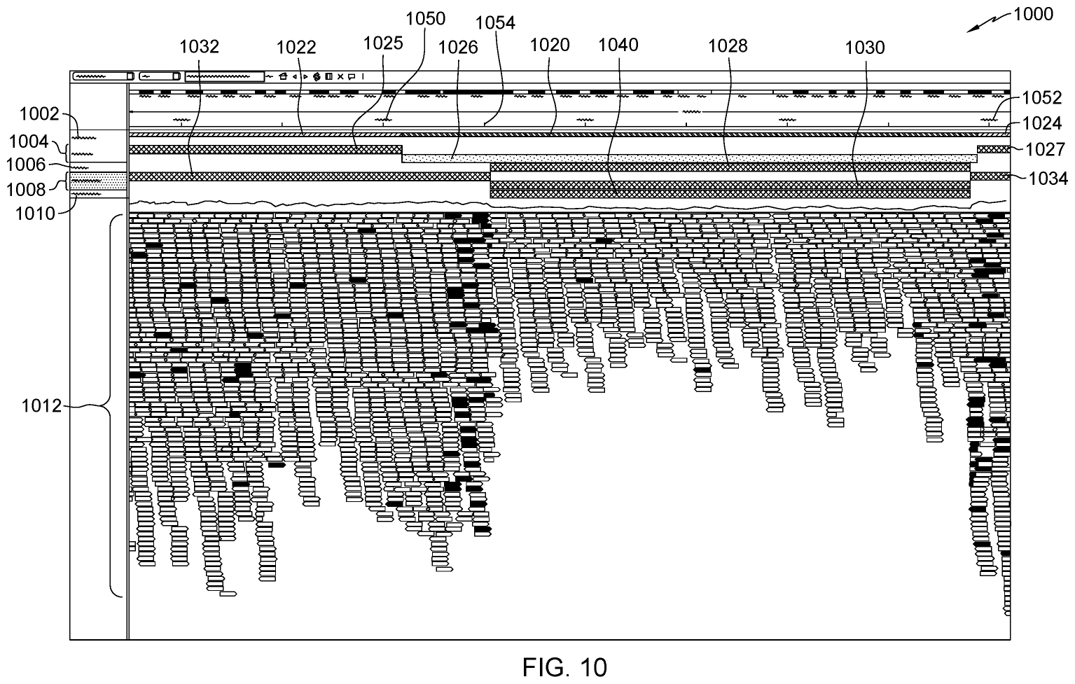

FIG. 10 presents an example interface 1000 illustrating a comparison between an SV-informed CNV call in accordance with aspects described herein and the associated CNV-only and SV-only calls from conventional CNV and SV call processing. The upper portion of the interface 1000 presents tracks 1002, 1004, 1006, 1008 and 1010 that visually represent variant call data (from VCF files for instance). Different portions of the tracks discussed with reference to FIGS. 10-12 herein may be presented in differing colors (or other visual characteristics) to distinguish between them. The lower portion 1012 of interface 1000 presents a binary alignment map (BAM file) pile-up representation of the read evidence (coverage and fluctuations thereof).

Track 1002, which may have a label such as “HG002.cnv.gff3”, is a features track. Track 1002 has a gap 1020 between portions 1022 and 1024 of the track indicating that the CNV call indicated by track 1004 was indicated as a Fail (filtered out) by the CNV caller software. In this case, the CNV indicated by track 1004 would not have been called as a CNV.

The other tracks 1004, 1006, 1008 and 1010 indicate where the corresponding events are located per the VCF file(s) with the data for each track. Track 1004, which may have a label such as “CNV (Original)”, is the CNV-only track, i.e., what the CNV caller (e.g. conventional CNV caller) alone would have called. Here, the portion 1026 of the track 1004 that is offset from the portions 1025, 1027 of the track 1004 indicates a CNV call that begins just past the 207,840 kbp position 1050 and ends just before the 207,846 kbp position 1052. Track 1006, which may have a label such as “SV (Original)”, is the SV-only track, i.e., what the SV caller (e.g. conventional SV caller) alone would have called. Here, portion 1028 indicates an SV call that begins just after the 207,841 kbp position 1054 and extends to just before where the original CNV call (or track 1004) ended, i.e., just before the 207,846 kbp position 1052. The SV call of track 1006 is noticeably shorter than the CNV call of track 1004. Track 1008 is, which may have a label such as “CNV (SV Adjusted)”, presents an SV-informed CNV call (CNV+SV call) determined in accordance with aspects described herein. Portion 1030 of the track 1008 that is offset from portions 1032 and 1034 of the track 1008 indicates an SV-informed CNV call that begins and ends where the SV call of track 1006 begins and ends. Track 1010 indicates, by portion 1040, the location of the particular variant as indicated by the NIST-provided truth set, i.e., a high confident call that was curated from the NIST consortia. It is seen that there is much better overlap of the CNV+SV call 1030 with the NIST truth 1040 as compared to the overlap of the original CNV call 1026 with the NIST truth 1040.

In the example of FIG. 10, the CNV caller identified the DEL but overcalled in that the start location is before the actual start location of the event and the end location is before the actual end location of the event. Meanwhile, the SV event called by the SV caller aligns well with the BAM pileup (as indicated by the vertical dashed lines), and the CNV+SV adjusted call of track 1008 mirrors what the SV call and truth indicate.

In this example, the original CNV call and original SV call can be matched and the breakpoints of the CNV aligned with those of the SV call that were observed by the SV caller. In this regard, the CNV+SV call is an SV-informed CNV call. Additionally, considering the CNV caller's capability and its output in isolation, the original CNF call was filtered out as indicated by track 1002. However, the CNV+SV call may be made with higher confidence such that the CNV call is made and the original CNV (as modified) is rescued/recovered (i.e., modified and reported rather than being filtered out as it would have been).

In conventional practice, SV callers and CNV callers each generate their own data files indicating SV and CNV call data, respectively. Typically, these follow the VCF specification with tab-separated fields. Some fields are standard, though some are not in order to provide flexibility for software tools to introduce caller-specific annotations and other information.

To illustrate an example of SV-informed CNV calling, consider the following example SV and CNV data/records, and the resulting SV-informed CNV call made based thereon. The example records were taken from the scenario of FIG. 9 in which the copy number loss and breakends indicated support a single DEL event. The call data/record for the SV call produced by the SV caller is as follows:

chr1 105095607 MantaDEL:61583:0:1:0:0:0 G <DEL>
34 PASS END=105167400;SVTYPE=DEL;SVLEN=−
71793;IMPRECISE; CIPOS=−416,417;CIEND=−232,233 GT:FT:GQ:PL:PR
0/1:PASS:34:84,0,564:37,7

The SV call data includes fields for presenting different information. chrl indicates chromosome 1 as an identifier of the chromosome in which the event appears. 105095607 indicates a start position of the event, i.e. where the variant is located. MantaDEL:61583:0:1:0:0:0 is caller-specific information and is not mandated by the VCF specification. Often this contains debug information for developers and is unused by the end user. G is the reference allele, meaning a G base should be found on chromosome 1 at the start position 105095607. <DEL> is the alternate allele or the variant that is being proposed by this record. Special notation of the VCF specification gives callers the ability to provide symbolic alleles—instead of listing out the full bases, the variant can be provided as a bracketed symbolic allele. 34 is a quality score indicator in phred scale, indicating a confidence level in the call. Typically a threshold is defined and a quality score must be above that threshold number for the call to Pass (insofar as confidence is concerned). The alternative is a Fail as a way of filtering out calls of low confidence. PASS is provided in a filter field and indicates that this SV call passed. If the record fails any filters (defined in the header of the VCF), this filter field can indicate such failure(s). Otherwise, as here, it indicates PASS.

The data following the PASS indication are informational annotations that may be defined in the header of the VCF file. END=105167400 indicates an end position of the event in question. SVTYPE=DEL is an informational field and the field indicates a deletion (“DEL” as here), duplication (“DUP”), etc. SVLEN=−71793 indicates the length of the structural variant in question. Deletion events are indicated by negative numbers and duplication events are indicated as positive numbers. IMPRECISE is indicated in a flag field to indicate whether the SV caller was able to confidently assemble the deletion event indicated. The IMPRECISE value here indicates that the call is not precise. CIPOS=−416,417 and CIEND=−232,233 are caller confidence intervals around the start position (CIPOS) and end position (CIEND) of the event.

GT:FT:GQ:PL:PR 0/1:PASS:34:84,0,564:37,7 present two columns of information—the first (GT:FT:GQ:PL:PR) provides colon-delimited abbreviations for values in corresponding positions of the second column (0/1:PASS:34:84,0,564:37,7). Thus, the GT abbreviation for genotype corresponds to the allele values 0/1, the FT abbreviation for sample filter applied at the sample level corresponds to PASS, the GQ abbreviation for genotype quality corresponds to the phred-scaled numeric value 34, the PL abbreviation correlates to the values 84,0,564 which are normalized phred-scaled likelihoods of the genotypes considered in the variant record for the sample, and the PR abbreviation corresponds to the improper pairs read counts numbers 37,7.

Next consider the following CNV call data/record for the CNV call provided by the CNV caller for the example of FIG. 9:

chr1 105095597 DRAGEN:LOSS:chr1:105095598-105167360 N
<DEL> 53 PASS SVLEN=−
71763;SVTYPE=CNV;END=105167360;REFLEN=71763 GT:SM:CN:BC:PE
0/1:0.47692:1:66:16,27

The example CNV call data includes some of the same fields as the SV call fields discussed above, for instance fields for a chromosome identifier, start position of the event, caller-specific information, the reference allele, variant indicator, quality score, and filter field indicating pass/fail status, as well as the SVLEN and SVTYPE fields. It also includes indicators END= and REFLEN= for end position and length of the CNV event.

The colon-delimited abbreviations (GT:SM:CN:BC:PE) again correspond to values (0/1:0.47692: 1:66: 16,27) that follow the abbreviations. The SM abbreviation for segment mean corresponds to 0.47692, the CN abbreviation for rounded copy number corresponds to value 1, and the BC and PE abbreviations correspond to values 66 and 12,27, respectively, which are algorithmic indicators of support for the CNV.

The above SV and CNV data records from the SV caller and CNV caller, respectively, can be correlated/matched in accordance with aspects described herein to form an SV-informed CNV call. The correlating/matching can be based on parsing SV and CNV VCF data produced by the respective callers, examining the breakpoints indicated for SV calls and CNV calls, and attempting to correlate/match SV calls to CNV calls. An SV call matching to a CNV call can inform of modifications to the CNV call to produce an SV-informed CNV call in accordance with aspects described herein.

Based on correlating/matching an SV call to a CNV call, a process can determine an SV-informed CNV call and provide corresponding VCF data to indicate this call. In examples, this may all be performed by software, for instance software that incorporates the SV caller and/or CNV caller, for instance the DRAGEN software offered by Illumina Inc. In examples, this VCF data for an SV-informed CNV call is generated based on modifying the data of the CNV call (or a copy thereof).

The following presents example VCF data that may be output for an SV-informed CNV call that was determined/composited from correlating an SV call (e.g., the “original SV call”—reflected by the SV call data above) and a CNV call (e.g., the “original CNV call”—reflected by the CNV call data above):

```
chr1 105095607 DRAGEN:LOSS:chr1:105095607-105167400 G
  <DEL> 53 PASS SVLEN=−
71793;SVTYPE=DEL;END=105167400;REFLEN=71793;SVCLAIM=DJ;IMPR
ECISE;CIPOS=−416,417;CIEND=−232,233 GT:SM:CN:BC:FT:GQ:PL:PR
  0/1:0.47692:1:66:PASS:34:84,0,564:37,7
```

The example SV-informed CNV call record above includes many of the same fields as the SV and CNV calls discussed above, with a mixture of annotations from each. This data includes fields for a chromosome identifier, start position of the event, caller-specific information, the reference allele, variant indicator, quality score, and filter field indicating pass/fail status, as well as the SVLEN and SVTYPE fields, and fields for position (END=) and event length (REFLEN=). In this example, the start and end positions of the CNV have been modified to be the start position (105095607) and end position (105167400) of the SV event. Other information that is dependent on these modifications, for instance event length, can also be updated. SVLEN of −71793 is used rather than the SVLEN −71763 of the original CNV record, for instance. Additionally, the quality score of 53 means that the filter result (PASS) remains the same as the result of the CNV record, but it is possible that if the filter result of the original CNV record was FAIL, this might be updated to PASS in the SV-informed CNV call record on the basis of determining this more accurate SV-informed CNV call. In some examples, an SV-informed CNV call is necessarily updated to indicate a PASS status (if the CNV call information did not already indicate PASS) and no change is made to the quality score. In other examples, the quality score can be modified to indicate a higher quality, i.e., at least to a minimum level to result in a PASS. In yet another example, the quality score is set to the maximum confidence level to ensure a PASS result.

The call also includes a field (e.g. SVCLAIM=DJ field here) to indicate the source of this call, for instance to distinguish between CNV only, SV only, and SV-informed (SV+CNV) CNV calls. In this example, the “DJ” attribute indicates use of depth (D) and junction (J) signals.

Thus, in determining an SV-informed CNV call, one or both endpoints of the call are expected to be updated/adjusted relative to the original position(s) indicated by the original CNV. Additionally, the quality score and value in the filter field may also be updated.

The call data of an SV-informed CNV call can be output just as call data of SV and CNV calls might be output, for instance in a VCF file. The VCF could be an existing VCF file that is produced (for instance a VCF file in which SV and/or CNV records are output), or a different VCF file. In some examples, the original CNV record would no longer be included in the final output VCF(s). This might be desired so that downstream analysis does not consider the original CNV record to itself indicate a CNV in addition to the SV-informed CNV. However, in some embodiments, the original SV VCF and CNV VCFs can still be made available (output), for instance to provide backward compatibility for tertiary analysis software.

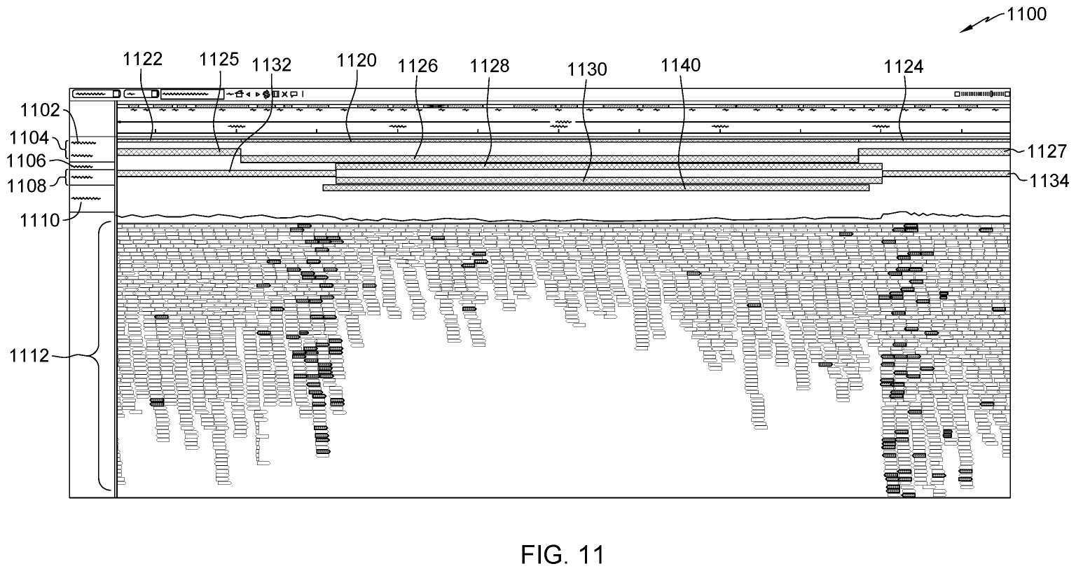

FIG. 11 presents another example interface 1100 illustrating a comparison between a structural-variant-informed copy number variant call in accordance with aspects described herein and the associated copy number variant-only and structural variant-only calls from conventional copy number variant and structural variant call processing. The comparison illustrates another example of adjustment to the boundaries of the original CNV call. Tracks 1102, 1104, 1106, 1108, and 1110 again visually represent variant call data while the lower portion 1112 of interface 1100 presents a BAM pile-up representation of the read evidence. Track 1102 is the features track with a portion 1120 between portions 1122 and 1124, which indicate that the CNV call indicated by track 1104 was indicated as a Pass by the CNV caller software. Track 1104 is the “CNV (Original)” track for the original CNV call with portion 1126 (offset from portions 1125, 1127 of track 1104) indicating the CNV call and start/end positions. Track 1106 is the “SV (Original)” track for the original SV call. Portion 1128 indicates an SV call that starts at a position significantly after the start position of the CNV (indicated by 1126) and ends at a position that is after the end position of the CNV. Track 1108 is the “CNV (SV Adjusted)” track presenting an SV-informed CNV call determined in accordance with aspects described herein. Portion 1130 of the track 1108 is offset from portions 1132 and 1134 of the track to indicate a CNV_SV call, which begins and ends where the SV call of track 1106 begins and ends. Compared to the ends of the original CNV (indicated by 1126), the ends of the SV-informed CNV call indicated by 1130 are moved inward on the front/start and out ends to match with the SV call (indicated by 1128). Track 1110 indicates via portion 1140 the location of the particular variant as indicated by the NIST-provided truth set at 1140. In this case, there is discrepancy between the endpoints of the SV-informed CNV call and the truth in that the SV-informed CNV appears shifted to a later start position, however the final (SV-informed CNV) call is still in much better agreement with the truth than the original CNV call, which spilled over significantly on the front end but passed the filtering and would have been reported by the CNV caller as a less accurate CNV than the SV-informed CNV.

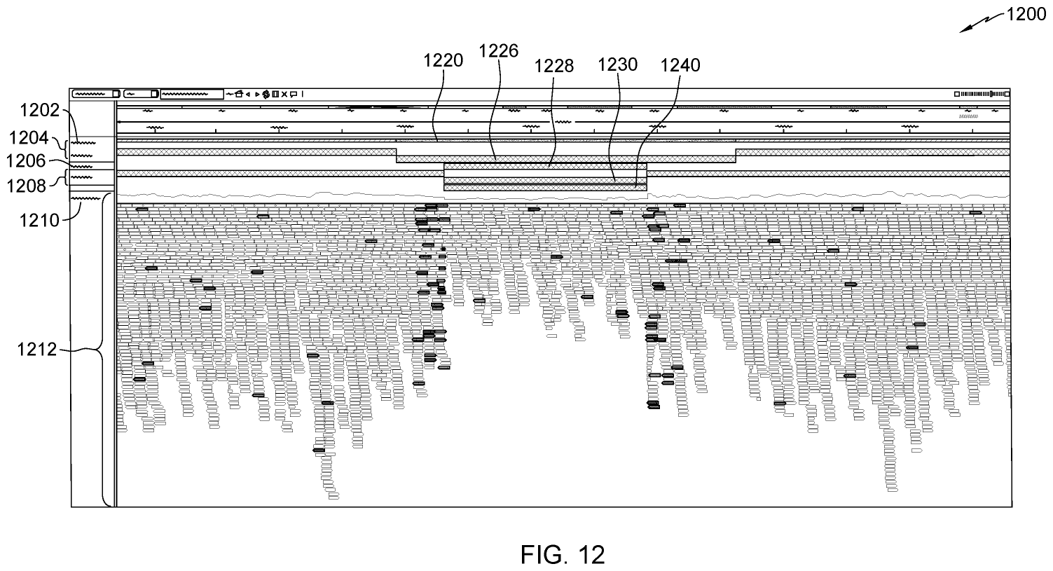

FIG. 12 presents a further example interface 1200 illustrating a comparison between a structural-variant-informed copy number variant call in accordance with aspects described herein and the associated copy number variant-only and structural variant-only calls from conventional copy number variant and structural variant call processing. Tracks 1202, 1204, 1206, 1208, and 1210 again represent the features track, “CNV (Original)” track, “SV (Original)” track, “CNV (SV Adjusted)” track, and NIST-provided truth set track, respectively. Lower portion 1212 of interface 1200 presents a BAM pile-up representation of the read evidence.

Portion 1220 of track 1202 may be presented in a color (such as grey) that indicates that the called original CNV record (indicated by 1226) was filtered out and would not have been reported. However, an adjustment as described herein adjust the breakpoints of the original CNV call (indicated by 1226) based on the breakpoints of the original SV call (indicated by 1228) to form the SV-informed CNV call (indicated by 1230), in which both breakpoints are moved inward (the start breakpoint of the CNV is moved to a greater base position and the end breakpoint is moved to a lesser base position) to match the SV. This results in an updated quality score and filter status to render the SV-informed CNV call a passing record, useful in downstream (e.g., tertiary) analysis against the event.

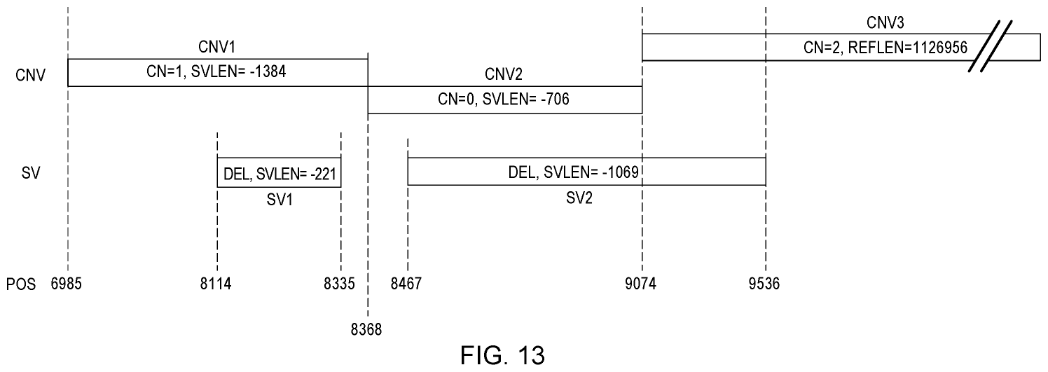

Further details of merging calls and dealing with conflicting breakpoints are now discussed with reference to FIG. 13, depicting an example conceptual diagram of CNV and SV calls along a segment of a genome. Shown are three adjacent CNV calls CNV1, CNV2 and CNV3, and two SV calls SV1 and SV2. Dashed vertical lines leading from the ends of the calls to position indicators/values (POS) below indicate the breakpoints associated with each of the calls.

In general, a process can correlate CNV and SV calls through their records, determining which, if any, SV calls match to any given CNV call, based on defined rules. Taking FIG. 13 as an example, first consider the following example raw records for the CNVs and SVs of FIG. 13:

−[CV1:] chr21 14086984 DRAGEN:LOSS:chr21:14086985-14088368 N
<DEL> 17 PASS SVLEN=−1384; SVTYPE=CNV; END=14088368;
REFLEN=1384 GT:SM:CN:BC:PE 0/1:0.429575:1:2:11,83
−[CNV2:] chr21 14088368 DRAGEN:LOSS:chr21:14088369-14089074 N
<DEL> 36 PASS SVLEN=−706; SVTYPE=CNV; END=14089074;
REFLEN=706 GT:SM:CN:BC:PE 1/1:1.00055e−09:0:1:83,67
−[CNV3:] chr21 14089075 DRAGEN:REF:chr21:14089075-15216030 N
. 54 PASS END=15216030; REFLEN=1126956 GT:SM:CN:BC:PE
./.:1.04001:2:2031:67,52
−[SV1:] chr21 14088114 MantaDEL:8384:19:20:0:0:0 AATTATATATA...
A 999 PASS END=14088335;SVTYPE=DEL;SVLEN=−221;
CIGAR=1M221D; CIPOS=0,1; HOMLEN=1; HOMSEQ=A
GT:FT:GQ:PL:PR:SR 1/1:PASS:95:999,98,0:0,0:0,32
−[SV2:] chr21 14088467 MantaDEL:8384:19:20:0:0:1 T <DEL> 999
PASS END=14089536;SVTYPE=DEL;SVLEN=−1069;CIPOS=0,1;CIEND=0,1;
HOMLEN=1;HOMSEQ=C GT:FT:GQ:PL:PR:SR
1/1:PASS:132:999,135,0:0,8:0,41

An example correlation process performs pairwise comparisons between each CNV and SV, and then for each breakpoint. In the following, the parenthetical notations indicate the start or end position of the given CNV event followed by the start or end of the given SV event, i.e., “(Start, End)” means the CNV Start, i.e., the coordinate (cnvCoord) of the CNV start, and the SV End, i.e., the coordinate (svCoord) of the SV end. A comparison of (X,Y) evaluates whether the breakpoint of the CNV should potentially be modified to be the breakpoint of the SV and checks orientation, relating to the change in depth status to ensure it is consistent (i.e., REF->DUP transition or REF->DEL transition, DEL->REF, DUP->REF. The term ‘orientation’ in this context pertains to depth status transition, and compatibility is important. For instance, if an SV breakpoint indicates a change from REF->DUP but the CNV breakpoint indicates REF->DEL, then these are incompatible and not be matched. The term “otherEndCoord” refers to the opposite end that is currently being evaluated. Thus, if evaluating a start position, then “otherEndCoord” refers to the stop position.

The example process's examination of CNV2 and CNV3 for potential SV-informed modification(s) thereof to form SV-informed CNV call(s) proceeds as follows: For CNV2:
Compare with SV1:
   (Start, Start)//compare CNV2 start to SV1 start
      This Fails because svCoord <svOtherEndCoord && svOtherEndCoord <=cnvCoord (the SV1 start is positionally before the SV1 end and the SV1 end is at or before the CNV2 start). This enforces a rule that requires at least some overlap between the SV and CNV as proposed to be modified.
   (Start, End)//compare CNV2 start to SV1 end
      This Fails on haveCompatibleOrientations (i.e., the CNV2 and SV1 would have incompatible orientations). In other words, CNV2 start is incompatible with SV1 end and cannot be updated as such because CNV2 start is the start of a DEL, while the SV1 end is the end of a DEL. This enforces a rule checking compatibility in orientations (of the proposed modified CNV and the SV).
   (End, Start)/compare CNV2 end to SV1 start
      Fails because cnvOtherEndCoord >=svCoord (the CNV2 start is positionally at or after the SV1 start). In other words, CNV2 End cannot be updated to be SV1 Start because CNV2 Start (which is to be positionally before the CNV End) is at/after the SV1 start, which would produce a non-positive-length event/variant.
   (End, End)//compare CNV2 end to SV1 end
      Fails because cnvOtherEndCoord >=svCoord (the CNV2 start is positionally at or after the SV1 end), which would produce a non-positive-length event/variant.
Compare with SV2:
   (Start, Start)//compare CNV2 start to SV2 start
      Passes (the two positions are within some threshold distance, such as 1,000 bp, for example) and is unique (a check ensures that a single breakpoint only matches one other breakpoint, i.e., is unambiguous). This enforces a rule for correlated breakpoints (e.g., CNV_start and SV_start here) to be within some threshold distance.
   (Start, End)//compare CNV2 start to SV2 end
      Fails on positionsAreCloseEnough (1168<=1000)
   (End, Start)//compare CNV2 end to SV2 start
      Fails on haveCompatibleOrientations (i.e., the CNV2 and SVs would have incompatible orientations, as CNV2 end is the end of a DEL, while the SV1 start is the start of a DEL).
   (End, End)//compare CNV2 end to SV2 end
      Passes (the two positions are within some threshold distance) and is unique

For CNV3:
Compare with SV1:
   (Start, Start)//compare CNV3 start to SV1 start
      Fails because svCoord <svOtherEndCoord && svOtherEndCoord <=cnvCoord (the SV1 start is positionally before the SV1 end and the SV1 end is at or before the CNV3 start)
   (Start, End)//compare CNV3 start to SV1 end
      Passes, and is the first hit so it is marked as unique
   (End, Start)//compare CNV3 end to SV1 start
      Fails on positionsAreCloseEnough (1127916<=1000)—the difference between CNV3 start and SV1 end is 1127916, which is not less than or equal to the threshold of 1000
   (End, End)//compare CNV3 end to SV1 end
      Fails on positionsAreCloseEnough (1127695<=1000)—the difference between the CNV3 end and SV1 end is 1127695, which is not less than or equal to the threshold of 1000
Compare with SV2:
   (Start, Start)//compare CNV3 start to SV2 start
      Fails on haveCompatibleOrientations (i.e., the CNV2 and SV1 would have incompatible orientations)
   (Start, End)//compare CNV3 start to SV2 end
      Passes, and now this is no longer marked as unique because the CNV3 start breakpoint matched also to SV1 end breakpoint.
   (End, Start)//compare CNV3 end to SV2 start
      Fails on positionsAreCloseEnough (1127563<=1000)—the difference between the CNV3 end and SV2 start is 1127563, which is not less than or equal to the threshold of 1000
   (End, End)//compare CNV3 end to SV2 end
      Fails on positionsAreCloseEnough (1126494<=1000)—the difference between the CNV3 end and SV2 end is 1126494, which is not less than or equal to the threshold of 1000

Based on the above, original CNV2 matches to original SV2 because of (i) the two passes on (Start, Start) and (End, End) and (ii) the CNV start and end each passing uniquely. Therefore, the SV-informed CNV call would call a CNV beginning where SV2 begins and ending where SV2 ends. Original CNV3 does not match to either of the original SV1 or SV2 and is not modified.

In this example, the end of CNV2 uniquely matched to the end of SV2 (and is therefore updated), but the start of CNV3 did not uniquely match to the end of SV2 (it also matched to SV1 start). CNV3 therefore does not get updated. This results in an overlapping segment of CNV2 and CNV3 since the end position of CNV2 is updated but start of CNV3 is not. In some examples if this is not desired, a potential workaround is to mitigate scenarios where conflicts happen by only allowing for SVs>minLength (say 500 bp) to be candidates for merging. Additionally or alternatively, if a previous CNV's end point is adjusted and it abuts the following CNV, then that following CNV can be force-adjusted to match the previous adjustment.

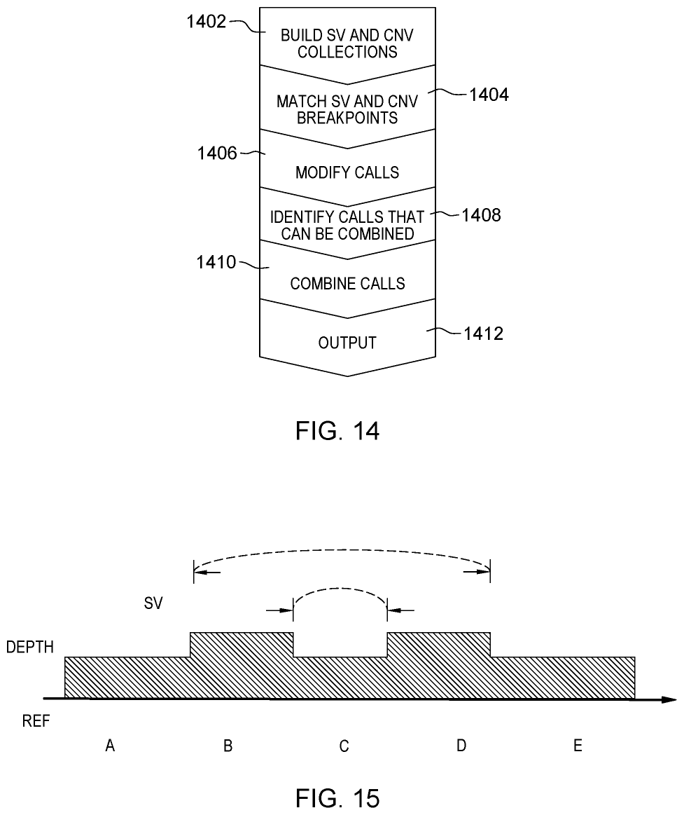

FIG. 14 depicts a conceptual diagram of a process for structural-variant-informed copy number variant calling and output, in accordance with aspects described herein. The process includes a step 1402 of building SV and CNV collections, which composes the original CNV and SV records with sv/cnv annotations. In embodiments, each SV and CNV record can be wrapped in a respective matchable structure, and such matchable structures for each given chromosome can be provided in a pos-sorted vector. The top-level collection can map contig names to the vectors.

The process of FIG. 14 also includes a step 1404 of matching SV and CNV breakpoints, for instance using proximity, orientation, and uniqueness rules discussed and exemplified herein to determine compatibility/correlation/matching. In examples, the nearest match wins. In example embodiments, CNV matching inquires whether a CNV start can be matched to an SV, and if so the respective matchable structure for the CNV records the index into an SV vector of the best-matching SV, as well as whether, in the case of DEL or TANDUP, the match is to the SV end. Similarly, the best match for the CNV end can be recorded into the matchable structure for the CNV, recording an index into an SV vector of the best-matching SV for the CNV end and whether the match is to the SV end.

The process of FIG. 14 continues with step 1406 to modifying calls. In examples, this is performed just for CNV calls by adjusting CNV endpoints, for instance by adding “LEFT_BND_OF” and “LEFT_BND” indicators for original CNV records (or copies thereof). CNV records with SV matches can be modified as follows: For the left-end SV match, adjust the CNV start position, add an origCnvPos annotation, and add a LEFT_BND annotation. For the right-end SV match, adjust the CNV end, add an origCnvEnd annotation, and add RIGHT_BND annotation.

Continuing with FIG. 14, the process includes steps 1408 and 1410 to identify calls that can be combined (1408) and combining them (1410). If Both ends of a CNV match corresponding ends of a compatible DEL or TANDUP, SV content can be added to a mergeable CNV. In a specific example, SV and CNV records are compressed/zipped into SvCnvMergedCall records that merge SV data into CNF call data when the calls are compatible. In examples, CNV calls are not ruled out based on lack of SV signal, meaning a CNV call made by the CNV caller will not be removed on the basis of a corresponding SV call/signal failing to be present for that CNV.

In some embodiments of the identify/combining calls steps (1408, 1410), the process modifies merged calls, where, for each SV-informed CNV call (SV+CNV call), for instance maintained in a combined call collection, the call adds the SV ID to the CNV record INFO and/or adds other SV INFO or FORMAT/sample content to the CNV record.

FIG. 14 also includes an output step 1412 where sorted records are written with a VCF file writer. In embodiments, SVs records that were not matched to any CNV record are written to a VCF file by rendering the SV records to a VCF string and parsing that string into a VCF record. Meanwhile, header lines from the CNV and SV record VCF file(s) can be combined into a single header, with deduplication of redundant content, and then each entry of a “combinedCalls” collection is output as an entry in a VCF, where entries that are CNV or SV+CNV are written to file by the tools used by the CNV caller, for instance a VCF record writer, and entries that are SV-only are rendered by an SV record renderer, the resulting string is converted to a VCF record, and is written to file by ta VCF record writer.

In examples, matching constraints used in the matching (1404) can include a maxCoordDelta (set at 2000, for example), which is a coarse level distance check to group together CNVs and SVs, and a maxCnvGap (set at 20000, for example), which is a parameter used to determine the transition state of a CNV between two CNVs. A distance larger than this value would mark the transition as unknown, since there may be other CNVs in between these two CNVs.

Example situations when ends of calls are allowed to match (i.e., as in step 1404) are as follows: coordinates have to be “close enough”, and no other compatible call is closer; close enough can be defined by a parameter indicating a number of base pairs, such as 2,000, direction of copy number change cannot conflict with SV “open”-sidedness—an increase in copy number conflicts with a right-open BND or the right-open side of a DEL/TDUP, a decrease in copy number conflicts with a left-open BND or the right-open side of a DEL/TDUP, and direction of copy number change may be considered “unknown” (allowed to match any SV) if there is a big enough gap (defined by a parameter indicating a number of base pairs, such as 20,000) between the relevant CNV end and the next-closest CNV end, and (iii) both the start and end pairs match, i.e. both CNV ends are matched to the same SV DEL or TDUP.

In relatively simple situations of 1-1 matching between a CNV and SV DEL or DUP, the records can be mergeable, in which SV record information is provided in the CNV record. Meanwhile, CNVs without related SVs can be left alone (reported as is), and short SV calls without compatible CNVs can be left alone. Aspects presented herein are also amenable to reconstruction of more complex structural variants as well, for instance in situations where a 1-1 match (CNV to SV) is not suitable. In these situations, CNV and SV records can remain separate but link to one another. In an example, the breakends of CNV records can be refined and each end of a CNV call can be linked to a breakend (if present). In an example, for large (germline and somatic) SV calls that are not directly equivalent to a called CNV, these can be decomposed into breakends, and these breakends can be linked to compatible CNV ends.

The following presents a scenario where a DELETION event is wholly contained within a DUPLICATION. Consider a depth and breakend profile of this situation as shown in FIG. 15. A depth-based CNV caller would detect this as two elevated duplication events—following from left to right (A-B-C-D-E): ‘ . . . REF-DUP-REF-DUP-REF . . . ’. The following presents SV and CNV (ignoring REF regions) records as were reported individually for this example scenario of FIGS. 15/16:

```
[SV]: 18 2634272 MantaDUP:TANDEM:154754:0:1:0:0:0 T
<DUP:TANDEM> 826 PASS END=2733679; SVTYPE=DUP;
SVLEN=99407; CIPOS=0,2; CIEND=0,2; HOMLEN=2; HOMSEQ=TT
GT:FT:GQ:PL:PR:SR 0/1:PASS:826:876,0,999:53,18:49,15
[SV]: 18 2643737 MantaDEL:154756:0:1:0:0:0 A <DEL> 758
PASS END=2655644; SVTYPE=DEL; SVLEN=−11907; SVINSLEN=3;
SVINSSEQ=ATC GT:FT:GQ:PL:PR:SR 0/1:PASS:758:808,0,999:47,16:50,14
[CNV]: 18 2633245 DRAGEN:GAIN:18:2633246-2643044 N
  <DUP> 37 cnvLength SVLEN=9799; SVTYPE=CNV;
END=2643044;REFLEN=9799 GT:SM:CN:BC:PE ./1:1.5916:3:8:33,24
[CNV]: 18 2655539 DRAGEN:GAIN:18:2655540-2733932 N
  <DUP> 58 PASS SVLEN=78393; SVTYPE=CNV; END=2733932;
REFLEN=78393 GT:SM:CN:BC:PE ./1:1.59787:3:71:23,27
```

This has the likely oversimplified explanation that there are just two CNV duplications occurring within this locus. This interpretation does not indicate what is more likely the true sequence of events to explain the construction of this locus—specifically, that there was a duplication across B-C-D and then a deletion within C. Factoring-in the SV BND signals, the start of B (or end of A) is tied to the end of D (or start of E), and that the start of C (or end of B) is tied to the end of C (or start of D).

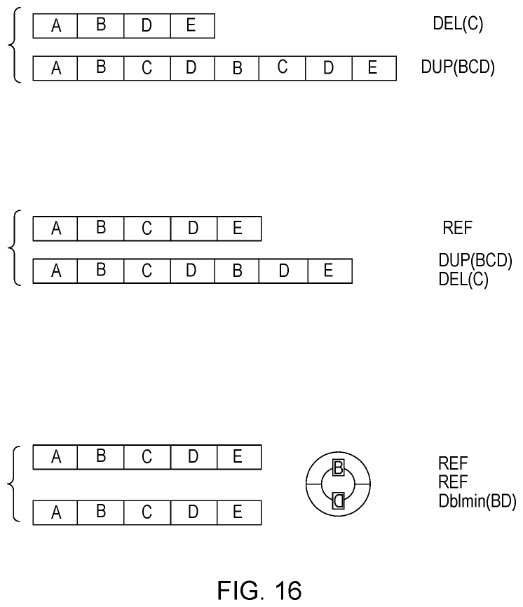

With the above, FIG. 16 presents some possible reconstructions of the event. It can be readily observed that, as to copy changes, they all share an extra copy of B and an extra copy of D, reflected as the following records, respectively:

18 2634272 DRAGEN:GAIN:18:2634272-2643737 N <DUP> 37 cnvLength
SVLEN=xxxx; SVTYPE=CNV; END=2643737; REFLEN=xxxx
GT:SM:CN:BC:PE ./1:1.5916:3:8:33,24
18 2655539 DRAGEN:GAIN:18:2655539-2733679 N <DUP> 58 PASS
SVLEN=xxxx; SVTYPE=CNV; END=2733679; REFLEN=xxxxx
GT:SM:CN:BC:PE ./1:1.59787:3:71:23,27

As to new adjacencies, the reconstructions shown in FIG. 16 present the following:
end(D) connected to beg(B)→
   beg(B) is preceded by end(D)
   end(D) is followed by beg(B)
end(B) connected to beg(D)→
   end(B) is followed by beg(D)
   beg(D) is preceded by end(B)

Note the last (third) scenario of FIG. 16 involves a “double minute”—small fragments of extrachromosomal DNA, which could explain the elevated depth within B and D.

The new adjacencies can be reflected by the following records (with original SV calls being decomposed into breakends):
[beg(B) is preceded by end(D)]:

18 2634272 MantaBND:1754:0:1:0:0:0 T ]18:2733679]T 826 PASS
SVTYPE=BND; MATEID=MantaBND:1754:0:1:0:0:1; CIPOS=0,2;
CIEND=0,2; HOMLEN=2; HOMSEQ=TT GT:FT:GQ:PL:PR:SR
0/1:PASS:826:876,0,999:53,18:49,15

[end(D) is followed by beg(B)]:

18 2733679 MantaBND:1754:0:1:0:0:1 C C[18:2634272[ 826 PASS
SVTYPE=BND;MATEID=MantaBND:1754:0:1:0:0:0; CIPOS=0,2; CIEND=0,2;
HOMLEN=2; HOMSEQ=TT GT:FT:GQ:PL:PR:SR
0/1:PASS:826:876,0,999:53,18:49,15

[end(B) is followed by beg(D)]:

18 2643737 MantaBND:154756:0:1:0:0:0 A A[18:2655539[ 758
PASS SVTYPE=BND; MATEID=MantaBND:1756:0:1:0:0:1; SVINSLEN=3;
SVINSSEQ=ATC GT:FT:GQ:PL:PR:SR 0/1:PASS:758:808,0,999:47,16:50,14

[beg(D) is preceded by end(B)]:

18 2655539 MantaBND:154756:0:1:0:0:1 G ]18:2643737]G 758
PASS SVTYPE=BND; MATEID=MantaBND:1756:0:1:0:0:0; SVINSLEN=3;
SVINSSEQ=ATC GT:FT:GQ:PL:PR:SR 0/1:PASS:758:808,0,999:47,16:50,14

At this point, processing can match the CNV start/end positions to the respective BNDs by updating the CNV records to reflect the “linkage” to the SV BND records. Meanwhile, the SV records, having been decomposed into their constituent BND formats, can be updated as well. The following presents updated CNV and SV records resulting from the extra copy of B, which entails that there is something other than A preceding a copy of B and something other than C following a copy of B (italics emphasize the notable properties for purposes of linkage):

−18 2634272 DRAGEN:GAIN:18:2634272-2643737 N <DUP> 37
cnvLength SVLEN=xxxx; SVTYPE=CNV; END=2643737; REFLEN=xxxx;
LEFT_{—}BND=MantaBND:1754:0:1:0:0:0;
RIGHT_{—}BND=MantaBND:1756:0:1:0:0:0 GT:SM:CN:BC:PE
./1:1.5916:3:8:33,24
−18 2634272 MantaBND:1754:0:1:0:0:0 T ]18:2733679]T 826 PASS
SVTYPE=BND;MATEID=MantaBND:1754:0:1:0:0:1;CIPOS=0,2;CIEND=0,2;
HOMLEN=2;HOMSEQ=TT; LEFT_{—}BND_{—}OF=DRAGEN:GAIN:18:2634272-
2643737 GT:FT:GQ:PL:PR:SR
0/1:PASS:826:876,0,999:53,18:49,15
−18 2643737 MantaBND:1756:0:1:0:0:0 A A[18:2655539[ 758 PASS
SVTYPE=BND; MATEID=MantaBND:1756:0:1:0:0:1; SVINSLEN=3;
SVINSSEQ=ATC; RIGHT_{—}BND_{—}OF=DRAGEN:GAIN:18:2634272-2643737
GT:FT:GQ:PL:PR:SR 0/1:PASS:758:808,0,999:47,16:50,14

In the above, LEFT_BND=MantaBND: 1754:0:1:0:0:0 and RIGHT_BND=MantaBND:1756:0:1:0:0:0 have been added to the first record to reflect the linkage to the second and third records. The second record has been modified to include MantaBND: 1754:0:1:0:0:0 and LEFT_BND_OF=DRAGEN:GAIN: 18:2634272-2643737 indicating the linkage to the first record as the left breakend. The third record has been modified to include MantaBND: 1756:0:1:0:0:0 and RIGHT_BND_OF=DRAGEN:GAIN: 18:2634272-2643737 indicating the linkage to the first record as the right breakend.

The following presents updated CNV and SV records resulting from the extra copy of D, which entails that there is something other than C preceding a copy of D and something other than A following a copy of D (italics emphasize the notable properties for purposes of linkage):

- 18 2655539 DRAGEN:GAIN:18:2655539-2733679 N <DUP> 58
PASS SVLEN=xxxx; SVTYPE-CNV; END-2733679; REFLEN=xxxxx;
LEFT_BND = MantaBND:1756:0:1:0:0:1; RIGHT_BND =
MantaBND:1754:0:1:0:0:1 GT:SM:CN:BC:PE ./1:1.59787:3:71:23,27
-18 2733679 MantaBND:1754:0:1:0:0:1 C C[18:2634272[ 826 PASS
SVTYPE=BND; MATEID=MantaBND:1754:0:1:0:0:0; CIPOS=0,2;
CIEND=0,2; HOMLEN=2; HOMSEQ=TT; RIGHT_BND_OF =
DRAGEN:GAIN:18:2655539-2733679 GT:FT:GQ:PL:PR:SR
0/1:PASS:826:876,0,999:53,18:49,15
-18 2655539 MantaBND:1756:0:1:0:0:1 G ]18:2643737]G 758 PASS
SVTYPE=BND; MATEID=MantaBND:1756:0:1:0:0:0; SVINSLEN=3;
SVINSSEQ=ATC; LEFT_BND_OF=DRAGEN:GAIN:18:2655539-2733679
GT:FT:GQ:PL:PR:SR 0/1:PASS:758:808,0,999:47,16:50,14

In the above, LEFT_BND=MantaBND: 1756:0:1:0:0:1 and RIGHT_BND=MantaBND:1754:0:1:0:0:1 have been added to the first record to reflect the linkage to the second and third records. The second record has been modified to include MantaBND: 1754:0:1:0:0:1 and RIGHT_BND_OF=DRAGEN:GAIN: 18:2655539-2733679 indicating the linkage to the first record as the right breakend. The third record has been modified to include MantaBND: 1756:0:1:0:0:1 and LEFT_BND_OF=DRAGEN:GAIN: 18:2655539-2733679 indicating the linkage to the first record as the left breakend.

The following presents the final calls that may be output based on the above, where the CNV calls have adjusted positions and linkages to BND calls, and the SVs are represented as BNDs, with linkages to the appropriate CNV call (italics emphasize the notable properties for purposes of linkage):

-18 2634272 DRAGEN:GAIN:18:2634272-2643737 N <DUP> 37
cnvLength SVLEN=xxxx; SVTYPE-CNV; END=2643737; REFLEN=xxxx;
LEFT_BND = MantaBND:1754:0:1:0:0:0; RIGHT_BND =
MantaBND:1756:0:1:0:0:0 GT:SM:CN:BC:PE ./1:1.5916:3:8:33,24
-18 2655539 DRAGEN:GAIN:18:2655539-2733679 N <DUP> 58
PASS SVLEN=xxxx; SVTYPE-CNV; END=2733679; REFLEN=xxxxx;
LEFT_BND = MantaBND:1756:0:1:0:0:1; RIGHT_BND =
MantaBND:1754:0:1:0:0:1 GT:SM:CN:BC:PE ./1:1.59787:3:71:23,27
-18 2634272 MantaBND:1754:0:1:0:0:0 T ]18:2733679]T 826 PASS
SVTYPE=BND; MATEID=MantaBND:1754:0:1:0:0:1; CIPOS=0,2;
CIEND=0,2; HOMLEN=2; HOMSEQ-TT; LEFT_BND_OF =
DRAGEN:GAIN:18:2634272-2643737 GT:FT:GQ:PL:PR:SR
0/1:PASS:826:876,0,999:53,18:49,15
-18 2733679 MantaBND:1754:0:1:0:0:1 C C[18:2634272[ 826 PASS
SVTYPE=BND; MATEID=MantaBND:1754:0:1:0:0:0; CIPOS=0,2;
CIEND=0,2; HOMLEN=2; HOMSEQ=TT;
RIGHT_BND_OF=DRAGEN:GAIN: 18:2655539-2733679
GT:FT:GQ:PL:PR:SR 0/1:PASS:826:876,0,999:53,18:49,15
-18 2643737 MantaBND:1756:0:1:0:0:0 A A[18:2655539[ 758 PASS
SVTYPE=BND; MATEID=MantaBND:1756:0:1:0:0:1; SVINSLEN=3;
SVINSSEQ=ATC; RIGHT_BND_OF=DRAGEN:GAIN:18:2634272-2643737
GT:FT:GQ:PL:PR:SR 0/1:PASS:758:808,0,999:47,16:50,14
-18 2655539 MantaBND:1756:0:1:0:0:1 G ]18:2643737]G 758 PASS
SVTYPE=BND; MATEID-MantaBND:1756:0:1:0:0:0; SVINSLEN=3;
SVINSSEQ=ATC; LEFT_BND_OF = DRAGEN:GAIN:18:2655539-2733679
GT:FT:GQ:PL:PR:SR 0/1:PASS:758:808,0,999:47,16:50,14

In comparison to the original two SV records, which indicated <DUP:TANDEM> and <DEL>, the updated records (four total) are presented as BND records in VCF format, which may be a preferred format for specifying complex rearrangements with breakends.

Based on the above, the CNVs will have updated breakpoints that are more accurate due to the finer resolution gained from leveraging the SV record information. It is noted that this set of records may not represent the entirety of the event—for instance REF record(s) may also be output as appropriate. In this example, the following REF record would be appropriate:

-18 2643737. DRAGEN:REF:18:2643737-2655539 N . 10 PASS
END=2655539; REFLEN=xxxx GT:SM:CN:BC:PE ./.:1.15766:2:11:24,23

It is noted that the above example output is just one example. Ultimately, how to report the event(s) may be determined based on the particular downstream interpretation tool(s) to be used and how they expect the event(s) to be presented.

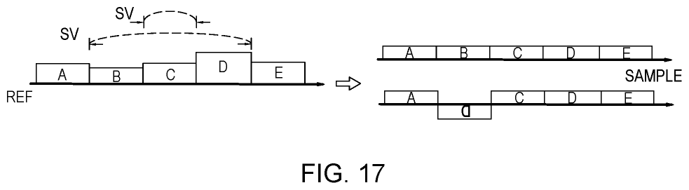

The following presents another complex scenario, this one involving a double-inverted deletion-duplication event. Consider a depth and breakend profile of this situation as shown in FIG. 17. The following presents the original SV records reported:

-16 34392108 MantaBND:151955:0:1:0:0:0:0 C C]16:34757737]
526 PASS SVTYPE=BND; MATEID=MantaBND:151955:0:1:0:0:0:1;
CIPOS=0,3; HOMLEN=3; HOMSEQ=TGG; BND_DEPTH=25;
MATE_BND_DEPTH=39 GT:FT:GQ:PL:PR:SR
0/1:PASS:526:576,0,659:31,12:34,9
-16 34757734 MantaBND: 151955:0:1:0:0:0:1 T T]16:34392111]
526 PASS SVTYPE=BND; MATEID=MantaBND:151955:0:1:0:0:0:0;
CIPOS=0,3; HOMLEN=3; HOMSEQ=CCA; BND_DEPTH=39;
MATE_BND_DEPTH=25 GT:FT:GQ:PL:PR:SR
0/1:PASS:526:576,0,659:31,12:34,9
-16 34396020 MantaBND:151962:0:1:0:0:0:1 A [16:34464610[A .
. SVTYPE=BND; MATEID=MantaBND:151962:0:1:0:0:0:0; IMPRECISE;
CIPOS=-257,258; BND_PAIR_COUNT=4; PAIR_COUNT=4
-16 34464610 MantaBND:151962:0:1:0:0:0:0 G [16:34396020[G .
. SVTYPE-BND; MATEID=MantaBND:151962:0:1:0:0:0:1; IMPRECISE;
CIPOS=-250,250; BND_PAIR_COUNT=6; PAIR_COUNT=4

In this example, only one of the two inversions is actually in the SV record call set (the other is only a candidate).

The following presents the original CNV records reported:

-16 34173661 DRAGEN:REF:16:34173661-34458728 N . 59
PASS END=34458728; REFLEN=285068 GT:SM:CN:BC:PE
./.:0.941707:2:226:20,1
-16 34466335 DRAGEN:GAIN:16:34466336-34757885 N
<DUP> 55 PASS SVLEN=291550; SVTYPE=CNV; END=34757885;
REFLEN=291550 GT:SM:CN:BC:PE ./1:1.43528:3:259:2,18
-16 34757886 DRAGEN:REF:16:34757886-35017793 N . 58
PASS END-35017793; REFLEN=259908 GT:SM:CN:BC:PE
./.:0.931997:2:218:18,19

In this example, there is no ‘DEL’ call corresponding to ‘B’ region in FIG. 17.

The following presents the final calls that may be output based on the above, where the CNV calls have adjusted positions and linkages to BND calls, and the SVs are represented as BNDs, with linkages to the appropriate CNV call (italics emphasize the notable properties for purposes of linkage):

-16 34173661 DRAGEN:REF:16:34173661-34458728 N . 59
PASS END-34458728; REFLEN=285068 GT:SM:CN:BC:PE
./.:0.941707:2:226:20,1
-16 34466335 DRAGEN:GAIN:16:34466336-34757885 N
<DUP> 55 PASS SVLEN=291550; SVTYPE=CNV; END-34757885;
REFLEN=291550; RIGHT_BND=MantaBND:151955:0:1:0:0:0:1
GT:SM:CN:BC:PE ./1:1.43528:3:259:2,18
-16 34757886 DRAGEN:REF:16:34757886-35017793 N . 58
PASS END-35017793; REFLEN=259908 GT:SM:CN:BC:PE
./.:0.931997:2:218:18,19
-16 34392108 MantaBND:151955:0:1:0:0:0:0 C C]16:34757737]
526 PASS SVTYPE=BND; MATEID=MantaBND: 151955:0:1:0:0:0:1;
CIPOS=0,3; HOMLEN=3; HOMSEQ=TGG; BND_DEPTH=25;
MATE_BND_DEPTH=39 GT:FT:GQ:PL:PR:SR
0/1:PASS:526:576,0,659:31,12:34,9
-16 34757734 MantaBND:151955:0:1:0:0:0:1 T T]16:34392111]
526 PASS SVTYPE=BND; MATEID=MantaBND:151955:0:1:0:0:0:0;
CIPOS=0,3; HOMLEN=3; HOMSEQ=CCA; BND_DEPTH=39;
MATE_BND_DEPTH=25; RIGHT_BND_OF=DRAGEN:GAIN:16:34466336-
34757885 GT:FT:GQ:PL:PR:SR 0/1:PASS:526:576,0,659:31,12:34,9

It is noted that in the above examples, there is no BND(_OF) for the candidate boundaries; only one end of the GAIN (second record above) has a BND. There is also no (short) CNV for one of the changes expected based on breakends; only one end of BND has a BND_OF (fifth record above). When there is a copy number change between x][x+1, if there is a breakend that corresponds to one side of the change point, this can be annotated on both of the CNV segments (i.e., RIGHT_BND for the segment ending at x and LEFT_BND for the segment starting at x+1). In other words, if an event is adjusted at one end, say positon x], then the adjacent event at [x+1 can also be adjusted and annotated, for instance in a later step to perform a pass-through of all records to ensure consistency.

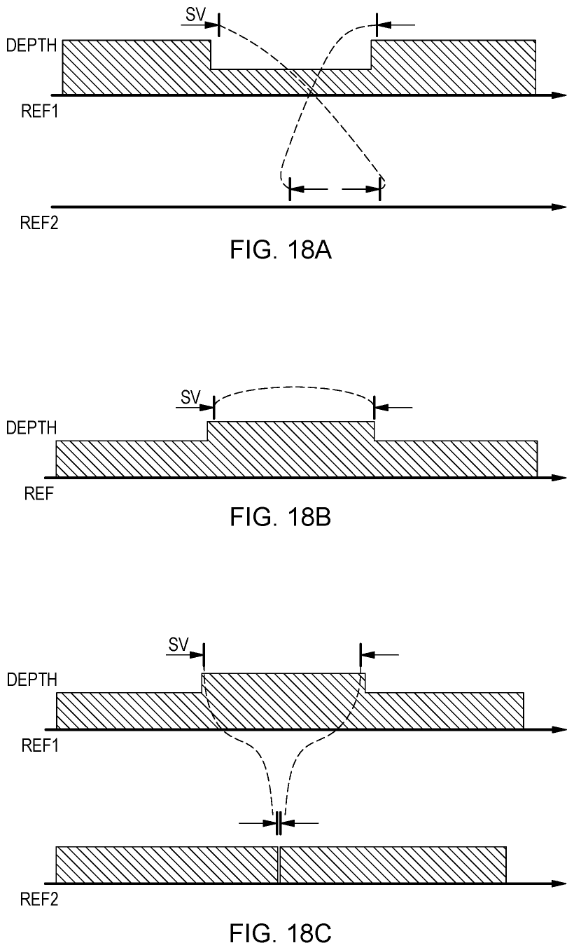

FIGS. 18A-18C and the corresponding description thereof provide additional examples in which SV-informed CNV calling is provided in accordance with aspects described herein.

FIG. 18A presents a deletion-based example in which the copy number loss and SV breakends do not support a DEL event. The original CNV record is as follows:

-chr1 105095597 DRAGEN:LOSS:chr5:105095598-105167360 N <DEL> 53
PASS SVLEN=-71763; SVTYPE-CNV; END=105167360; REFLEN=71763
GT:SM:CN:BC:PE 0/1:0.47692:1:66:16,27

The original SV records are as follows:

-chr1 105095607 MantaBND:57476:0:2:0:0:0:0 G G]chr2:105167960] 17
MinQUAL SVTYPE=BND; MATEID=MantaBND:57476:0:2:0:0:0:1;
CIPOS=0,2; HOMLEN=2; HOMSEQ=GT; BND_DEPTH=43;
MATE_BND_DEPTH=31 GT:FT:GQ:PL:PR:SR
0/1:PASS:17:67,0,999:49,4:29,5
-chr2 105167960 MantaBND:57476:0:2:0:0:0:1 A A]chr1:110417996] 17
MinQUAL SVTYPE=BND; MATEID-MantaBND:57476:0:2:0:0:0:0;
CIPOS=0,2; HOMLEN=2; HOMSEQ=TG; BND_DEPTH=31;
MATE_BND_DEPTH=43 GT:FT:GQ:PL:PR:SR
0/1:PASS:17:67,0,999:49,4:29,5
-chr1 105167460 MantaBND:1234:0:2:0:0:0:0 T [chr2:105130000[T 17
MinQUAL SVTYPE=BND; MATEID=MantaBND:1234:0:2:0:0:0:1;
CIPOS=0,2; HOMLEN=2; HOMSEQ=GT; BND_DEPTH=43;
MATE_BND_DEPTH=31 GT:FT:GQ:PL:PR:SR
0/1:PASS:17:67,0,999:49,4:29,5
-chr2 105130000 MantaBND:1234:0:2:0:0:0:1 C [chr1:105167460[C 17
MinQUAL SVTYPE-BND; MATEID=MantaBND:1234:0:2:0:0:0:0;
CIPOS=0,2; HOMLEN=2; HOMSEQ=TG; BND_DEPTH=31;
MATE_BND_DEPTH=43 GT:FT:GQ:PL:PR:SR
0/1:PASS:17:67,0,999:49,4:29,5

The following presents example records (indicating an SV-informed CNV call) that may be generated and output based on the above, and in accordance with aspects described herein:

-chr1 105095607 DRAGEN:LOSS:chr5:105095608-105167460 N <DEL> 53 PASS
SVLEN=-71763;SVTYPE-CNV;END-105167460;REFLEN=71763;
SVCLAIM=DJ; LEFT_BND=MantaBND:57476:0:2:0:0:0;
RIGHT_BND=MantaBND:1234:0:2:0:0:0:0 GT:SM:CN:BC:PE
0/1:0.47692:1:66:16,27
-chr1 105095607 MantaBND:57476:0:2:0:0:0:0 G G]chr2:105167960] 17
MinQUAL SVTYPE=BND; MATEID=MantaBND:57476:0:2:0:0:0:1;
CIPOS=0,2; HOMLEN=2; HOMSEQ=GT; BND_DEPTH=43;
MATE_BND_DEPTH=31; LEFT_BND_OF=DRAGEN:LOSS:chr5:105095608-
105167460 GT:FT:GQ:PL:PR:SR 0/1:PASS:17:67,0,999:49,4:29,5
-chr2 105167960 MantaBND:57476:0:2:0:0:0:1 A AJchr1:110417996] 17
MinQUAL SVTYPE=BND; MATEID=MantaBND:57476:0:2:0:0:0:0;
CIPOS=0,2; HOMLEN=2; HOMSEQ=TG; BND_DEPTH=31;
MATE_BND_DEPTH=43 GT:FT:GQ:PL:PR:SR
0/1:PASS:17:67,0,999:49,4:29,5
-chr1 105167460 MantaBND:1234:0:2:0:0:0:0 T [chr2:105130000[T 17
MinQUAL SVTYPE=BND; MATEID=MantaBND:1234:0:2:0:0:0:1;
CIPOS=0,2; HOMLEN=2; HOMSEQ=GT; BND_DEPTH=43;
MATE_BND_DEPTH=31; RIGHT_BND_OF=DRAGEN:LOSS:chr5:105095608-
105167460 GT:FT:GQ:PL:PR:SR 0/1:PASS:17:67,0,999:49,49,5
-chr2 105130000 MantaBND:1234:0:2:0:0:0:1 C [chr1:105167460[C 17
MinQUAL SVTYPE=BND; MATEID=MantaBND:1234:0:2:0:0:0:0;
CIPOS=0,2; HOMLEN=2; HOMSEQ=TG; BND_DEPTH=31;
MATE_BND_DEPTH=43 GT:FT:GQ:PL:PR:SR
0/1:PASS:17:67,0,999:49,4:29,5

In the above example, the “LEFT_BND” and “RIGHT_BND” link the records from CNV to BND, and the “LEFT_BND_OF” and “RIGHT_BND_OF” link the records from BND to CNV.

FIG. 18B presents a duplication-based example in which the duplication is supported by both CNV and tandup breakends. The original SV record is as follows:

-chr8 46613324 MantaDUP:TANDEM:125619:0:1:0:0:0 G
<DUP:TANDEM>407 PASS END-46633279; SVTYPE=DUP; SVLEN=19955;
CIPOS=0,9; CIEND=0,9; HOMLEN=9; HOMSEQ=ATGAGATAC
GT:FT:GQ:PL:PR:SR 0/1:PASS:407:457,0,999:42,11:39,7

The original CNV records are as follows:

-chr8 46495807 DRAGEN:REF:chr8:46495807-46612106 N . 48 PASS
END-46612106; REFLEN=116300 GT:SM:CN:BC:PE ./.:0.9589:2:98:15,3
-chr8 46614258 DRAGEN:GAIN:chr8:46614259-46629419 N <DUP> 30 PASS
SVLEN=15161; SVTYPE=CNV; END=46629419; REFLEN=15161
GT:SM:CN:BC:PE ./1:1 .45628:3:14:1,2
-chr8 46634456 DRAGEN:REF:chr8:46634456-60677768 N . 103 PASS
END-60677768; REFLEN=14043313 GT:SM:CN:BC:PE
./.:0.995336:2:12220:0,1

Note that there are gaps between flanking REF regions and the DUP. Matching criteria (e.g., proximity of end points) may need to be relaxed in this situation when a CNV boundary is next to a gap in (good) bin coverage.

The following presents an example record (indicating an SV-informed CNV call) that may be generated and output based on the above, and in accordance with aspects described herein:

-chr8 46613324 MantaDUP:TANDEM:125619:0:1:0:0:0 G <DUP:TANDEM>
407 PASS END=46633279; SVTYPE=DUP; SVLEN=19955; CIPOS=0,9;
CIEND=0,9; HOMLEN=9; HOMSEQ=ATGAGATAC
GT:FT:GQ:PL:PR:SR:SM:CN:BC:PE
0/1:PASS:407:457,0,999:42,11:39,7:1.45628:3:14:1,2

FIG. 18C depicts an example of a dispersed duplication (2nd non-tandem copy in genome). The original SV records are as follows:

-chr3 91357394 MantaBND:30982:0:1:0:0:0:0 T T[chr8:123907[746 PASS
SVTYPE=BND; MATEID=MantaBND:30982:0:1:0:0:0:1; CIPOS=0,2;
HOMLEN=2; HOMSEQ-GT; BND_DEPTH-53; MATE_BND_DEPTH=21
GT:FT:GQ:PL:PR:SR 0/1:PASS:746:796,0,830:37,4:31,23
-chr8 123907 MantaBND:30982:0:1:0:0:0:1 A ]chr3:51009231]A 746 PASS
SVTYPE=BND; MATEID=MantaBND:30982:0:1:0:0:0:0; CIPOS=0,2;
HOMLEN=2; HOMSEQ=GT; BND_DEPTH=21; MATE_BND_DEPTH=53
GT:FT:GQ:PL:PR:SR 0/1:PASS:746:796,0,830:37,4:31,23
-chr3 91357396 MantaBND:43623:0:1:0:0:0:0 A ]chr8:189442[GA 103 PASS
SVTYPE=BND; MATEID=MantaBND:43623:0:1:0:0:0:1; SVINSLEN=1;
SVINSSEQ-G; BND_DEPTH=42; MATE_BND_DEPTH=33
GT:FT:GQ:PL:PR:SR 0/1:PASS:103:153,0,936:36,0:5,7
-chr8 189442 MantaBND:43623:0:1:0:0:0:1 C CG[chr3:91357396[ 103 PASS
SVTYPE=BND; MATEID=MantaBND:43623:0:1:0:0:0:0; SVINSLEN=1;
SVINSSEQ=G; BND_DEPTH-33; MATE_BND_DEPTH=42
GT:FT:GQ:PL:PR:SR 0/1:PASS:103:153,0,936:36,0:35,7

The original CNV record is as follows:

-chr8 124109 DRAGEN:GAIN:chr8:124110-189632 N <DUP> 1000 PASS
SVLEN=65533; SVTYPE-CNV; END-189632; REFLEN=65533
GT:SM:CN:BC:PE ./1:1.45628:3:57:39,41

The following presents example records (indicating an SV-informed CNV call) that may be generated and output based on the above, and in accordance with aspects described herein:

-chr8 123907 DRAGEN:GAIN:chr8:123907-189442 N <DUP> 1000 PASS
SVLEN=65535; SVTYPE-CNV; END=189442; REFLEN=65535;
LEFT_BND=MantaBND:30982:0:1:0:0:0:1;
RIGHT_BND=MantaBND:43623:0:1:0:0:0:1 GT:SM:CN:BC:PE
./1:1.45628:3:57:39,41
-chr3 91357394 MantaBND:30982:0:1:0:0:0:0 T T[chr8:123907[746 PASS
SVTYPE=BND; MATEID=MantaBND:30982:0:1:0:0:0:1; CIPOS=0,2;
HOMLEN=2; HOMSEQ=GT; BND_DEPTH=53; MATE_BND_DEPTH=21;
LEFT_BND_OF=DRAGEN:GAIN:chr8:123907-189442GT:FT:GQ:PL:PR:SR
0/1:PASS:746:796,0,830:37,4:31,23
-chr8 123907 MantaBND:30982:0:1:0:0:0:1 A ]chr3:51009231]A 746 PASS
SVTYPE=BND; MATEID=MantaBND:30982:0:1:0:0:0:0; CIPOS=0,2;
HOMLEN=2; HOMSEQ=GT; BND_DEPTH=21; MATE_BND_DEPTH=53;
LEFT_BND_OF=DRAGEN:GAIN:chr8:123907-189442 GT:FT:GQ:PL:PR:SR
0/1:PASS:746:796,0,830:37,4:31,23
-mchr3 91357396 MantaBND:43623:0:1:0:0:0:0 A ]chr8:189442[GA 103 PASS
SVTYPE=BND; MATEID-MantaBND:43623:0:1:0:0:0:1; SVINSLEN=1;
SVINSSEQ=G;BND_DEPTH=42; MATE_BND_DEPTH=33;
RIGHT_BND_OF=DRAGEN:GAIN:chr8:123907-189442
GT:FT:GQ:PL:PR:SR 0/1:PASS:103:153,0,936:36,0:35,7
-chr8 189442 MantaBND:43623:0:1:0:0:0:1 C CG[chr3:91357396[ 103 PASS
SVTYPE=BND; MATEID=MantaBND:43623:0:1:0:0:0:0; SVINSLEN=1;
SVINSSEQ=G; BND_DEPTH=33;M ATE_BND_DEPTH=42;
RIGHT_BND_OF=DRAGEN:GAIN:chr8:123907-189442
GT:FT:GQ:PL:PR:SR 0/1:PASS:103:153,0,936:36,0:35,7

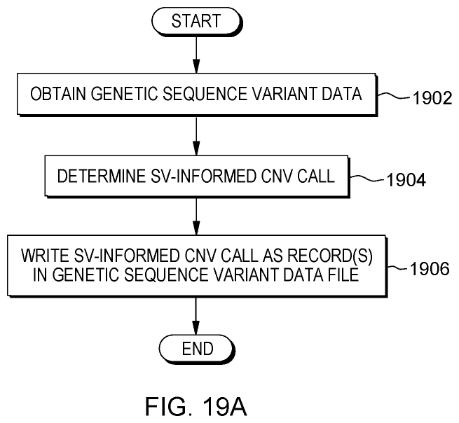

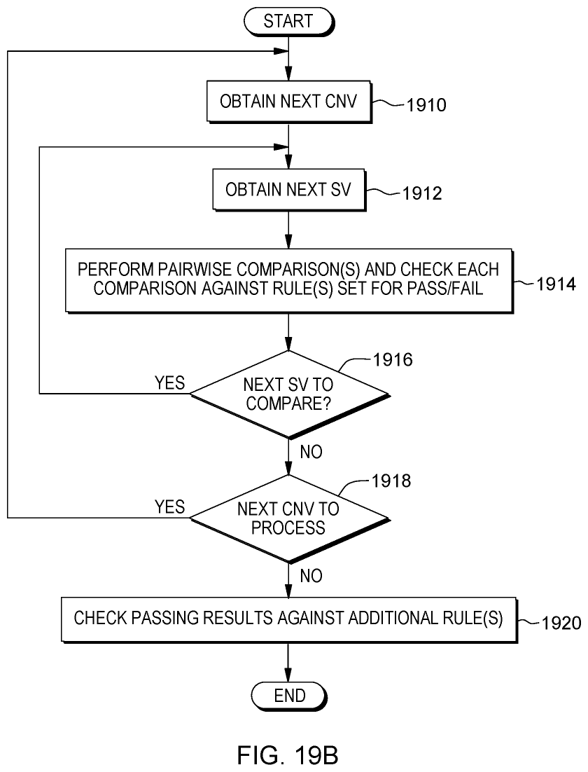

FIGS. 19A-19B depict example processes for improved calling of copy number variants in a genomic sequence, in accordance with aspects described herein. The processes may be executed, in one or more examples, by a processor or processing circuitry of one or more computers/computer systems, such as those described herein, which may include one or more computer systems providing/executing genomic analysis software, for instance.

Referring initially to FIG. 19A, the process obtains (1902) genetic sequence variant data that includes records indicating SV(s) and records indicating CNVs in the genomic sequence. The method also determines (1904), based on an initial CNV indicated in the genetic sequence variation data and on at least one initial SV indicated in the genetic sequence variation data, an SV-informed CNV call as an updated version of the initial CNV. This determining uses information from the at least one initial SV call to determine a start breakpoint position and an end breakpoint position for the SV-informed CNV call. At least one of the start breakpoint position and end breakpoint position are updated, informed by the at least one initial SV, in comparison to a corresponding start breakpoint position and/or end breakpoint position of the initial CNV.

In examples, the initial CNV and rest of the CNVs indicated by the records are provided by a CNV calling component of genomic analysis software. The genomic analysis software can have a filtering component configured to filter-out CNVs of which a confidence level is less than a threshold confidence level, and the CNV calling component can provides a confidence level of the initial CNV that is less than the threshold confidence level (i.e., the software would ordinarily filter this out). However, the SV-informed CNV call may be provided with a confidence level higher than the threshold confidence level such that the filtering component does not filter-out the SV-informed CNV call.

In examples, a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit. In examples, a breakpoint resolution of the SV-informed CNV call is 1 base pair.

The determining (1904) can be performed as part of a process for determining one or more SV-informed CNV calls based on the records obtained at 1902. An example such process is presented by FIG. 19B. The process of FIG. 19B runs through a set of one or more CNVs and a set of one or more SVs, performing a pairwise comparison of each CNV of the set to each SV of the set and applying rule(s) for determining whether to modify breakpoints of the CNV(s) based on SV data. The process of FIG. 19B begins by entering a first loop that cycles through each CNV of the set of CNV(s). Entrance into the loop beings by obtaining (1910) a next CNV of the set to process. The process then enters a second loop that cycles through each SV of the set of SV(s) by obtaining (1912) a next SV. The process then performs (1914) a pairwise comparison of that CNV to that SV. The pairwise comparison of the CNV to the SV includes breakpoint comparison(s) that each compare a respective first breakpoint position, of the CNV, to a respective second breakpoint position, of the SV, and checks/evaluates rule(s) for pass/failure based on the respective first breakpoint position being proposed for modification to be the respective second breakpoint position to provide a proposed modified CNV (a modified version of the CNV under processing). The breakpoint comparison(s) can include (i) comparing a start breakpoint position of the CNV to the start and/or end breakpoint positions of the SV, and/or (ii) comparing an end breakpoint position of the CNV to the start and/or end breakpoint positions of the SV. The rule(s) can include any desired rules for dictating situations when modifying a breakpoint position of the CNV to be a breakpoint position of an SV might be appropriate. Example such rules can includes, but are not limited to, a rule requiring at least some positional overlap between the SV and the proposed modified CNV, a rule for compatibility in orientation of the SV and the proposed modified CNV, and/or a rule for correlated breakpoints of the SV and proposed modified CNV to be within a threshold distance. A pass at this point is a provisional pass, as one or more additional rules may be checked during downstream processing (discussed below with respect to aspect 1920 of FIG. 19B).

After performing the pairwise comparison(s) at 1914, the process proceeds by determining (1916) whether there is any next SV of the set to compare to the CNV, i.e., whether there are any SVs of the set that have not undergone a comparison with this CNV. If so (1916, Y), the process iterates in the second loop by returning to 1912 to obtain a next SV and proceeding to performing (1914) the pairwise comparison(s) for the CNV and the next SV. This loop (1912, 1914, 1916 repeats until all SVs have been processed for this CNV. At that point, there are no next SVs to compare (1916, N) and the process proceeds by determining (1918) whether there are any next CNVs of the set of CNVs to process, i.e., whether there are any CNVs of the set that have not undergone comparison processing to the SVs of the set of SVs. If so (1918, Y), the process iterates in the first loop by returning to 1910 to obtain a next CNV, then proceeds to enter the second loop again by obtaining (1912) a next SV to process.

It is seen that the first loop iterates over the set of CNVs. Once all CNVs have been processed in this manner, there are no next CNVs to process (1918, N). At that point, there may be one or more passing results of the comparisons. Each passing result proposes to modify a respective breakpoint position of a CNV to be a respective breakpoint position of an SV. At this point, the process proceeds by checking (1920) the passing results against any applicable additional rule(s). By way of example, an additional rule might be a check to for uniqueness that, in order to pass, requires that any breakpoint of a CNV proposed for modification match to at most one SV breakpoint of the SV(s) of the set. Violation of this rule or any other additional rule(s) checked at 1920 might render an initially-passing result to instead fail. Otherwise, passing results will inform breakpoint modifications to make to initial CNV call data of the obtained records, in order to process one or more SV-informed CNV calls.

In some examples, determining an SV-informed CNV call as an updated version of an initial CNV includes modifying a record of the initial CNV to produce a record of the SV-informed CNV call, where the modifying changes the start and/or end breakpoint positions of the initial CNV, as indicated in the record of the initial CNV, to be the determined updated breakpoint position(s) informed by SV(s) of the set to which the initial CNV was compared (in FIG. 19B). As part of this, the process can also update a length of the CNV (indicated in the record of the initial CNV) and a quality score in the record of the initial CNV as appropriate to produce the data for the SV-informed CNV call. In examples, the record of the initial CNV (the record being modified to match the SV data) is a copy of an original record of the initial CNV. In examples, the genetic sequence variant data file that is obtained at 1902 is part of one or more genetic sequence variant data files, and the original record of the initial CNV is retained and output in at least one of those genetic sequence variant data file(s).

In some examples, a length of the SV-informed CNV call is less than or equal to 20,000 base pairs. In some examples, a length of the SV-informed CNV call is less than or equal to 10,000 base pairs. In some examples, a length of the SV-informed CNV call is less than a length of the initial CNV. In some examples, the length of the initial CNV is less than or equal to 20,000 base pairs.

Returning to FIG. 19A, with the SV-informed CNV call determined (1904), the process proceeds by writing (1906) the determined SV-informed CNV call as one or more records in a genetic sequence variant data file. It is noted that the process of FIG. 18A focuses on one SV-informed CNV call, though the determining and writing aspects of FIG. 18A can be performed for multiple SV-informed CNV calls in examples.

A sampling of aspects described herein is as follows:
A1. A computer-implement method for improved calling of copy number variants in a genomic sequence, the method comprising: obtaining genetic sequence variant data comprising records indicating at least one structural variant (SV) and records indicating at least one copy number variant (CNV) in the genomic sequence; determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and writing the determined SV-informed CNV call as one or more records in a genetic sequence variant data file.
A2. The method of A1, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.
A3. The method of A1, wherein a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit, and wherein a breakpoint resolution of the SV-informed CNV call is 1 base pair.
A4. The method of A1, wherein the determining the SV-informed CNV call as the updated version of the initial CNV comprises modifying a record of the initial CNV to produce a record of the SV-informed CNV call, the modifying changing the start and/or end breakpoint positions of the initial CNV, as indicated in the record of the initial CNV, to be the determined updated breakpoint position(s) informed by the at least one initial SV, and further updating a length of the CNV indicated in the record of the initial CNV and a quality score in the record of the initial CNV, to provide the record of the SV-informed CNV call.
A5. The method of A4, wherein the record of the initial CNV is a copy of an original record of the initial CNV, wherein the genetic sequence variant data file is part of one or more genetic sequence variant data files, and wherein the original record of the initial CNV is retained and output in at least one of the one or more genetic sequence variant data files.
A6. The method of A1, A2, A3, A4, or A5, wherein the determining the SV-informed CNV call comprises performing, for each initial SV of the at least one initial SV, a pairwise comparison of the initial CNV to the initial SV.
A7. The method of A6, wherein the pairwise comparison of the initial CNV to the initial SV comprises one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.
A8. The method of A7, wherein the one or more breakpoint comparisons comprise at least one of: comparing a start breakpoint position of the initial CNV to at least one of the start breakpoint position or end breakpoint position of the initial SV; or comparing an end breakpoint position of the initial CNV to at least one of the start breakpoint position or end breakpoint position of the initial SV.
A9. The method of A7, wherein the one or more rules comprise at least one of: a rule requiring at least some positional overlap between the initial SV and the proposed modified CNV; a rule for compatibility in orientation of the initial SV and the proposed modified CNV; a rule for correlated breakpoints of the initial SV and proposed modified CNV to be within a threshold distance; or a rule for uniqueness requiring that a breakpoint, of the initial CNV, proposed for modification match to at most one SV breakpoint of the at least one initial SV.
A10. The method of A1, A2, A3, A4, or A5, wherein a length of the SV-informed CNV call is less than or equal to 20,000 base pairs.
A11. The method of A1, A2, A3, A4, or A5, wherein a length of the SV-informed CNV call is less than or equal to 10,000 base pairs.
A12. The method of A1, A2, A3, A4, or A5, wherein a length of the SV-informed CNV call is less than a length of the initial CNV.
A13. The method of A12, wherein the length of the initial CNV is less than or equal to 20,000 base pairs.
B1. A computer system comprising: a memory; and a processor in communication with the memory, wherein the computer system is configured to perform a method for improved calling of copy number variants in a genomic sequence, the method comprising: obtaining genetic sequence variant data comprising records indicating at least one structural variant (SV) and records indicating at least one copy number variant (CNV) in the genomic sequence; determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and writing the determined SV-informed CNV call as one or more records in a genetic sequence variant data file.
B2. The computer system of B1, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.
B3. The computer system of B1, wherein a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit, and wherein a breakpoint resolution of the SV-informed CNV call is 1 base pair.
B4. The computer system of B1, wherein the determining the SV-informed CNV call as the updated version of the initial CNV comprises modifying a record of the initial CNV to produce a record of the SV-informed CNV call, the modifying changing the start and/or end breakpoint positions of the initial CNV, as indicated in the record of the initial CNV, to be the determined updated breakpoint position(s) informed by the at least one initial SV, and further updating a length of the CNV indicated in the record of the initial CNV and a quality score in the record of the initial CNV, to provide the record of the SV-informed CNV call.
B5. The computer system of B4, wherein the record of the initial CNV is a copy of an original record of the initial CNV, wherein the genetic sequence variant data file is part of one or more genetic sequence variant data files, and wherein the original record of the initial CNV is retained and output in at least one of the one or more genetic sequence variant data files.
B6. The computer system of B1, B2, B4, B4, or B5, wherein the determining the SV-informed CNV call comprises performing, for each initial SV of the at least one initial SV, a pairwise comparison of the initial CNV to the initial SV.
B7. The computer system of B6, wherein the pairwise comparison of the initial CNV to the initial SV comprises one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.
B8. The computer system of B7, wherein the one or more breakpoint comparisons comprise at least one of: comparing a start breakpoint position of the initial CNV to at least one of the start breakpoint position or end breakpoint position of the initial SV; or comparing an end breakpoint position of the initial CNV to at least one of the start breakpoint position or end breakpoint position of the initial SV.
B9. The computer system of B7, wherein the one or more rules comprise at least one of: a rule requiring at least some positional overlap between the initial SV and the proposed modified CNV; a rule for compatibility in orientation of the initial SV and the proposed modified CNV; a rule for correlated breakpoints of the initial SV and proposed modified CNV to be within a threshold distance; or a rule for uniqueness requiring that a breakpoint, of the initial CNV, proposed for modification match to at most one SV breakpoint of the at least one initial SV.
B10. The computer system of B1, B2, B4, B4, or B5 wherein a length of the SV-informed CNV call is less than or equal to 20,000 base pairs.
B11. The computer system of B1, B2, B4, B4, or B5, wherein a length of the SV-informed CNV call is less than or equal to 10,000 base pairs.
B12. The computer system of B1, B2, B4, B4, or B5, wherein a length of the SV-informed CNV call is less than a length of the initial CNV.
B13. The computer system of B12, wherein the length of the initial CNV is less than or equal to 20,000 base pairs.
C1. A computer program product comprising: a computer readable storage medium readable by a processing circuit and storing instructions for execution by the processing circuit for performing a method for improved calling of copy number variants in a genomic sequence, the method comprising: obtaining genetic sequence variant data comprising records indicating at least one structural variant (SV) and records indicating at least one copy number variant (CNV) in the genomic sequence; determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and writing the determined SV-informed CNV call as one or more records in a genetic sequence variant data file.
C2. The computer program product of C1, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.
C3. The computer program product of C1, wherein a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit, and wherein a breakpoint resolution of the SV-informed CNV call is 1 base pair.
C4. The computer program product of C1, wherein the determining the SV-informed CNV call as the updated version of the initial CNV comprises modifying a record of the initial CNV to produce a record of the SV-informed CNV call, the modifying changing the start and/or end breakpoint positions of the initial CNV, as indicated in the record of the initial CNV, to be the determined updated breakpoint position(s) informed by the at least one initial SV, and further updating a length of the CNV indicated in the record of the initial CNV and a quality score in the record of the initial CNV, to provide the record of the SV-informed CNV call.
C5. The computer program product of C4, wherein the record of the initial CNV is a copy of an original record of the initial CNV, wherein the genetic sequence variant data file is part of one or more genetic sequence variant data files, and wherein the original record of the initial CNV is retained and output in at least one of the one or more genetic sequence variant data files.
C6. The computer program product of C1, C2, C3, C4, or C5, wherein the determining the SV-informed CNV call comprises performing, for each initial SV of the at least one initial SV, a pairwise comparison of the initial CNV to the initial SV.
C7. The computer program product of C5, wherein the pairwise comparison of the initial CNV to the initial SV comprises one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.
C8. The computer program product of C7, wherein the one or more breakpoint comparisons comprise at least one of: comparing a start breakpoint position of the initial CNV to at least one of the start breakpoint position or end breakpoint position of the initial SV; or comparing an end breakpoint position of the initial CNV to at least one of the start breakpoint position or end breakpoint position of the initial SV.
C9. The computer program product of C7, wherein the one or more rules comprise at least one of: a rule requiring at least some positional overlap between the initial SV and the proposed modified CNV; a rule for compatibility in orientation of the initial SV and the proposed modified CNV; a rule for correlated breakpoints of the initial SV and proposed modified CNV to be within a threshold distance; or a rule for uniqueness requiring that a breakpoint, of the initial CNV, proposed for modification match to at most one SV breakpoint of the at least one initial SV.
C10. The computer program product of C1, C2, C3, C4, or C5, wherein a length of the SV-informed CNV call is less than or equal to 20,000 base pairs.
C11. The computer program product of C1, C2, C3, C4, or C5, wherein a length of the SV-informed CNV call is less than or equal to 10,000 base pairs.
C12. The computer program product of C1, C2, C3, C4, or C5, wherein a length of the SV-informed CNV call is less than a length of the initial CNV.
C13. The computer program product of C12, wherein the length of the initial CNV is less than or equal to 20,000 base pairs.

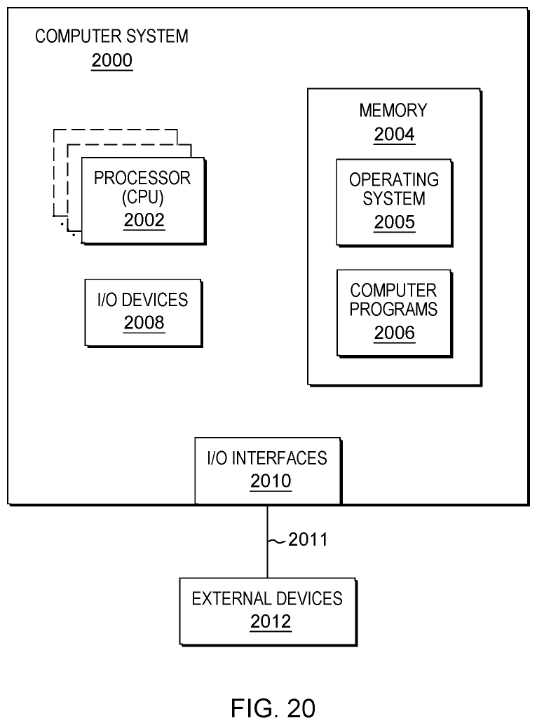

Processes described herein may be performed singly or collectively by one or more computer systems, such as one or more computer system(s) executing genomic analysis software to perform aspects described herein. FIG. 20 depicts an example of a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) as an example. FIG. 20 shows a computer system 2000 in communication with external device(s) 2012. Computer system 2000 includes one or more processor(s) 2002, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 2002 can also include register(s) to be used by one or more of the functional components. Computer system 2000 also includes memory 2004, input/output (I/O) devices 2008, and I/O interfaces 2010, which may be coupled to processor(s) 2002 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 2004 can be or include main or system memory (e.g. Random Access Memory) used in the execution of program instructions, storage device(s) such as hard drive(s), flash media, or optical media as examples, and/or cache memory, as examples. Memory 2004 can include, for instance, a cache, such as a shared cache, which may be coupled to local caches (examples include L1 cache, L2 cache, etc.) of processor(s) 2002. Additionally, memory 2004 may be or include at least one computer program product having a set (e.g., at least one) of program modules, instructions, code or the like that is/are configured to carry out functions of embodiments described herein when executed by one or more processors.

Memory 2004 can store an operating system 2005 and other computer programs 2006, such as one or more computer programs/applications that execute to perform aspects described herein. Specifically, programs/applications can include computer readable program instructions that may be configured to carry out functions of embodiments of aspects described herein.

Examples of I/O devices 2008 include but are not limited to microphones, speakers, Global Positioning System (GPS) devices, cameras, lights, accelerometers, gyroscopes, magnetometers, sensor devices configured to sense light, proximity, heart rate, body and/or ambient temperature, blood pressure, and/or skin resistance, and activity monitors. An I/O device may be incorporated into the computer system as shown, though in some embodiments an I/O device may be regarded as an external device (2012) coupled to the computer system through one or more I/O interfaces 2010.

Computer system 2000 may communicate with one or more external devices 2012 via one or more I/O interfaces 2010. Example external devices include a keyboard, a pointing device, a display, and/or any other devices that enable a user to interact with computer system 2000. Other example external devices include any device that enables computer system 2000 to communicate with one or more other computing systems or peripheral devices such as a printer. A network interface/adapter is an example I/O interface that enables computer system 2000 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 2010 and external devices 2012 can occur across wired and/or wireless communications link(s) 2011, such as Ethernet-based wired or wireless connections. Example wireless connections include cellular, Wi-Fi, Bluetooth®, proximity-based, near-field, or other types of wireless connections. More generally, communications link(s) 2011 may be any appropriate wireless and/or wired communication link(s) for communicating data.

Particular external device(s) 2012 may include one or more data storage devices, which may store one or more programs, one or more computer readable program instructions, and/or data, etc. Computer system 2000 may include and/or be coupled to and in communication with (e.g. as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 2000 may be operational with numerous other general purpose or special purpose computing system environments or configurations. Computer system 2000 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g. instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C #, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implement method for improved calling of copy number variants in a genomic sequence, the method comprising:
obtaining genetic sequence variant data comprising records indicating at least one structural variant (SV) and records indicating at least one copy number variant (CNV) in the genomic sequence;
determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and
writing the determined SV-informed CNV call as one or more records in a genetic sequence variant data file.

2. The method of claim 1, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.

3. The method of claim 1, wherein a breakpoint resolution of breakpoints of the initial CNV is equal to a window size of n base pairs, where 2000>n>200, such that a position of a breakpoint of the initial CNV is an approximated position identified based on a window, of length n, in which the breakpoint of the initial CNV is determined to sit, and wherein a breakpoint resolution of the SV-informed CNV call is 1 base pair.

4. The method of claim 1, wherein the determining the SV-informed CNV call as the updated version of the initial CNV comprises modifying a record of the initial CNV to produce a record of the SV-informed CNV call, the modifying changing the start and/or end breakpoint positions of the initial CNV, as indicated in the record of the initial CNV, to be the determined updated breakpoint position(s) informed by the at least one initial SV, and further updating a length of the CNV indicated in the record of the initial CNV and a quality score in the record of the initial CNV, to provide the record of the SV-informed CNV call.

5. The method of claim 4, wherein the record of the initial CNV is a copy of an original record of the initial CNV, wherein the genetic sequence variant data file is part of one or more genetic sequence variant data files, and wherein the original record of the initial CNV is retained and output in at least one of the one or more genetic sequence variant data files.

6. The method of claim 1, wherein the determining the SV-informed CNV call comprises performing, for each initial SV of the at least one initial SV, a pairwise comparison of the initial CNV to the initial SV.

7. The method of claim 6, wherein the pairwise comparison of the initial CNV to the initial SV comprises one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.

8. The method of claim 7, wherein the one or more breakpoint comparisons comprise at least one of:
comparing a start breakpoint position of the initial CNV to at least one of the start breakpoint position or end breakpoint position of the initial SV; or
comparing an end breakpoint position of the initial CNV to at least one of the start breakpoint position or end breakpoint position of the initial SV.

9. The method of claim 7, wherein the one or more rules comprise at least one of:
a rule requiring at least some positional overlap between the initial SV and the proposed modified CNV;
a rule for compatibility in orientation of the initial SV and the proposed modified CNV;
a rule for correlated breakpoints of the initial SV and proposed modified CNV to be within a threshold distance; or
a rule for uniqueness requiring that a breakpoint, of the initial CNV, proposed for modification match to at most one SV breakpoint of the at least one initial SV.

10. The method of claim 1, wherein a length of the SV-informed CNV call is less than or equal to 20,000 base pairs.

11. The method of claim 1, wherein a length of the SV-informed CNV call is less than or equal to 10,000 base pairs.

12. The method of claim 1, wherein a length of the SV-informed CNV call is less than a length of the initial CNV.

13. The method of claim 12, wherein the length of the initial CNV is less than or equal to 20,000 base pairs.

14. A computer system comprising:
a memory; and
a processor in communication with the memory, wherein the computer system is configured to perform a method for improved calling of copy number variants in a genomic sequence, the method comprising:
obtaining genetic sequence variant data comprising records indicating at least one structural variant (SV) and records indicating at least one copy number variant (CNV) in the genomic sequence;
determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and
writing the determined SV-informed CNV call as one or more records in a genetic sequence variant data file.

15. The computer system of claim 14, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.

16. The computer system of claim 14, wherein the determining the SV-informed CNV call as the updated version of the initial CNV comprises modifying a record of the initial CNV to produce a record of the SV-informed CNV call, the modifying changing the start and/or end breakpoint positions of the initial CNV, as indicated in the record of the initial CNV, to be the determined updated breakpoint position(s) informed by the at least one initial SV, and further updating a length of the CNV indicated in the record of the initial CNV and a quality score in the record of the initial CNV, to provide the record of the SV-informed CNV call.

17. The computer system of claim 14, wherein the determining the SV-informed CNV call comprises performing, for each initial SV of the at least one initial SV, a pairwise comparison of the initial CNV to the initial SV, and wherein the pairwise comparison of the initial CNV to the initial SV comprises one or more breakpoint comparisons that each compare a respective first breakpoint position, of the initial CNV, to a respective second breakpoint position, of the initial SV, by evaluating one or more rules for pass/failure based on the respective first breakpoint position being proposed for modification to be the respective second breakpoint position to provide a proposed modified CNV.

18. The computer system of claim 17, wherein the one or more rules comprise at least one of:
a rule requiring at least some positional overlap between the initial SV and the proposed modified CNV;
a rule for compatibility in orientation of the initial SV and the proposed modified CNV;
a rule for correlated breakpoints of the initial SV and proposed modified CNV to be within a threshold distance; or
a rule for uniqueness requiring that a breakpoint, of the initial CNV, proposed for modification match to at most one SV breakpoint of the at least one initial SV.

19. A computer program product comprising:
a computer readable storage medium readable by a processing circuit and storing instructions for execution by the processing circuit for performing a method for improved calling of copy number variants in a genomic sequence, the method comprising:
obtaining genetic sequence variant data comprising records indicating at least one structural variant (SV) and records indicating at least one copy number variant (CNV) in the genomic sequence;
determining, based on an initial CNV indicated in the genetic sequence variant data and on at least one initial SV indicated in the genetic sequence variant data, an SV-informed CNV call as an updated version of the initial CNV, wherein the determining uses information from the at least one initial SV to determine a start breakpoint position and an end breakpoint position for the SV-informed CNV call, at least one of the start breakpoint position and end breakpoint position being updated, informed by the at least one initial SV, in comparison to a corresponding start breakpoint position and/or end breakpoint position of the initial CNV; and
writing the determined SV-informed CNV call as one or more records in a genetic sequence variant data file.

20. The computer program product of claim 19, wherein the initial CNV is provided by a CNV calling component of genomic analysis software, wherein the genomic analysis software has a filtering component configured to filter-out CNVs of which a confidence level is less than a threshold confidence level, wherein the CNV calling component provides a confidence level of the initial CNV that is less than the threshold confidence level, and wherein the SV-informed CNV call is provided with a confidence level higher than the threshold confidence level such that the filtering component does not filter-out the SV-informed CNV call.
